(19) United States
(12) Patent Application Publication
(10) Pub. No.: US 2010/0330704 A1
(43) Pub. Date: Dec. 30, 2010

(54) COMPOSITE PARTICLE, METHOD FOR PRODUCING THE SAME, DISPERSION SOLUTION, MAGNETIC BIOSENSING APPARATUS AND MAGNETIC BIOSENSING METHOD

(75) Inventors: Kazumichi Nakahama, Tokyo (JP); Miki Ueda, Tokyo (JP); Akira Kuriyama, Atsugi-shi (JP); Takayuki Hiratani, Tokyo (JP); Ikuo Nakazawa, Kawasaki-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/865,063

(22) PCT Filed: Jun. 6, 2009
(86) PCT No.: PCT/JP2009/061066

§ 371 Date: Jul. 28, 2010

(30) Foreign Application Priority Data
Jun. 12, 2008 (JP) 2008-154646

(51) Int. Cl. G01N 33/543

(52) U.S. Cl. 436/518; 422/69

(57) ABSTRACT

To provide a method for producing composite particles small in particle size, excellent in mono-dispersibility, high in magnetic-substance content per particle, large in saturation magnetization, excellent in dispersion stability and having non-specific adsorption suppressibility.

The method includes (1) mixing a first liquid and particles to prepare a mixture solution;(2) mixing the mixture solution and a second liquid to prepare an emulsion containing a dispersoid formed of the first liquid and the particles; (3) mixing a polymer compound with the emulsion; and (4) fractionating the emulsion to extract the first liquid from the dispersoid to produce the composite particles each containing the particles and the polymer compound, characterized in that the dispersoid has a single-peak particle size distribution and a dispersity index (Dhw/Dhn) calculated from a number-average hydrodynamic particle size (Dhn) and a weight-average hydrodynamic particle size (Dhw) is 1.5 or less.

### TECHNICAL FIELD

The present invention relates to composite particles, a method for producing the same, a dispersion solution, a magnetic biosensing apparatus and a magnetic biosensing method.

### BACKGROUND ART

Recently, research and development have been aggressively made on composite particles directed to application to various industrial fields. For example, magnetic particles, which are formed of a polymer compound and a magnetic substance, are expected to have a wide variety of uses. Particularly, attention has been focused on uses of a base material in the field of medical/diagnosis such as medicinal drugs and diagnostic agents.

As uses of magnetic particles in the medical/diagnosis field, for example, a magnetic biosensor may be mentioned. The magnetic biosensor, which is one of the highly sensitive sensing systems recently proposed, detects the presence/absence or concentration of a target substance in a test solution by detecting the presence/absence or the number of magnetic particles positioned in the proximity of the surface of a detection site.

As examples of the magnetic biosensor, SQUID (superconducting quantum interference device), a hole-effect element, a magnetic resistant effect element and a magnetic impedance element, etc. are known.

The magnetic particles used in a magnetic biosensor are suitable if they have the following three characteristics: (1) being small in particle size and excellent in mono-dispersibility; (2) being high in content of a magnetic substance and large in saturation magnetization per particle; and (3) being excellent in dispersion stability.

The above characteristic (1) “being small in particle size and excellent in mono-dispersibility” is connected to improvement of detection speed of a magnetic biosensor for a target substance and quantitativity thereof. Furthermore, the characteristic (2) “being high in content of a magnetic substance and large in saturation magnetization per particle” is connected to improvement of detection sensitivity to a target substance. The characteristic (3) “being excellent in dispersion stability” is connected to improvement of detection sensitivity to a target substance and reproducibility thereof.

However, in most cases, the above three characteristics are in a trade-off relationship. Therefore, it is difficult to produce magnetic particles satisfying all characteristics. In the context of the aforementioned conditions, magnetic particles formed of a non-magnetic substance such as a polymer compound, which has a relatively high degree of freedom in molecular design and selectivity, and a magnetic substance, have been reported.

Japanese Patent Application Laid-Open No. 2004-099844 discloses an approach for obtaining magnetic particles formed of a polymer compound and a magnetic substance by use of a mini-emulsion polymerization method. In addition, a method for obtaining magnetic particles formed of a polymer compound and a magnetic substance by use of a soap-free emulsion polymerization method is known.

In the approaches using the mini-emulsion polymerization method and the soap-free emulsion polymerization method, it is contemplated that saturation magnetization is increased by enhancing the content of the magnetic substance in magnetic particles. However, for application to a magnetic biosensor, the magnitude of saturation magnetization per magnetic substance is not yet sufficient. What is excellent in these approaches is that the dispersion stability of magnetic particles is successfully improved by coating the surface of the magnetic particles thickly with a non-magnetic substance, i.e., a polymer compound.

Generally, in the magnetic particles having a certain level of saturation magnetization or more, the dispersion stability significantly decreases due to a stray magnetic field leaking from the magnetic particles. Then, to improve the dispersion stability of the magnetic particles, it is generally believed that it is effective to employ a method of reducing the stray magnetic field leaking from the magnetic particles by providing a coating layer formed of a non-magnetic substance to the magnetic particles.

However, the magnetic biosensor detects the presence/absence or the number of magnetic particles by detecting the stray magnetic field leaking from the magnetic particles as a signal. Therefore, providing the coating layer formed of a non-magnetic substance as mentioned above to the magnetic particles is not preferable since the stray magnetic field leaking from the magnetic particles is reduced.

On the other hand, in the magnetic biosensor, it is known that non-specific adsorption of the magnetic particles to a non-target substance increases noise and decreases reproducibility. Then, it has been desired to develop magnetic particles having the aforementioned three characteristics and none or less occurrence of non-specific adsorption. As a potential method for reducing non-specific adsorption, a method of coating the surface of particles with a material having non-specific adsorption suppressibility has been proposed. However, the magnetic particles produced by the approaches using the mini-emulsion polymerization method and the soap-free emulsion polymerization method has a thick coating layer formed of a non-magnetic substance on the surface. Therefore, providing a coating layer of a material having non-specific adsorption suppressibility further on the thick coating layer is disadvantageous in detecting the stray magnetic field of the magnetic particles. For the reason, it has been desired to develop magnetic particles having the aforementioned three characteristics with the thinnest possible coating layer formed of a non-magnetic substance thereon.

On the other hand, as quantitative immunoassay, RIA (radio immunoassay) and IRMA (immunoradiometric assay) have long been known. In this assay, a competitive antigen or antibody is labeled with a radionuclide and the specific radioactivity is measured. Based on the measurement results, the antigen is quantitatively determined. In short, a target substance, such as an antigen etc., is labeled and measured indirectly. This method has a high sensitivity and thus made a great contribution in clinical diagnosis. However, the safety of radionuclide must be taken into consideration and thus a specific plant and apparatus are required. Then, as a method easier to handle, methods employing a label made of a fluorescent substance, an enzyme, an electrochemiluminescence molecule and a composite particle such as a magnetic particle have been proposed.

When a label such as a fluorescent label, an enzyme label or an electrochemiluminescence label is used, an optical measurement method is employed. A target substance is detected by measuring light absorption, transmittance or light-emission amount. EIA (Enzyme Immunoassay) using an enzyme as a label is a colorimetric method which includes reacting an antigen and an antibody followed by an enzyme-labeled antibody, adding the substrate of the enzyme to emit a color and determining quantity based on absorbance.

Furthermore, researches on a biosensor for detecting a biomolecule indirectly by using a magnetic particle as a label and a magnetic sensor device have been reported by some research institutes. As the magnetic sensor device used in this detection method, various types of devices may be mentioned. For example, a device using a magnetoresistance effect element, a device using a hole element, a device using Josephson element, a device using a coil, a device using an element whose magnetic impedance changes and a device using a flux gate element have been reported.

As described above, research and development have recently been aggressively made on composite particles directed to application to various industrial fields, particularly, in the field of medical/diagnosis.

### DISCLOSURE OF THE INVENTION

The present invention was made in view of the background art mentioned above and is directed to providing composite particles small in particle size, excellent in mono-dispersibility, high in magnetic-substance content per particle, large in saturation magnetization, excellent in dispersion stability and having non-specific adsorption suppressibility, and a method of producing the same.

Furthermore, the present invention is directed to providing a dispersion solution using the above composite particles.

Moreover, the present invention is directed to providing a magnetic biosensing apparatus using the composite particles and a biosensing method.

More specifically, the present invention is directed to method for producing composite particles including

(1) mixing a first liquid and particles to prepare a mixture solution;

(2) mixing the mixture solution and a second liquid to prepare an emulsion containing a dispersoid formed of the first liquid and the particles;

(3) mixing a polymer compound with the emulsion; and

(4) fractionating the emulsion to extract the first liquid from the dispersoid to produce the composite particles each containing the particles and the polymer compound, characterized in that the dispersoid has a single-peak particle size distribution and a dispersity index (Dhw/Dhn) calculated from a number-average hydrodynamic particle size (Dhn) and a weight-average hydrodynamic particle size (Dhw) is 1.5 or less.

Furthermore, the present invention provides composite particles each having a structure in which substantially spherical multinuclear particles formed of a plurality of magnetic substances are surrounded by a film-state polymer compound, characterized in that a polydispersity index (Dw/Dn) calculated from a number-average dry particle size (Dn) and a weight-average dry particle size (Dw) of the composite particles is 1.2 or less; the weight-average dry particle size (Dw) of the composite particles falls within the range of 50 nm to 300 nm; the content of the magnetic substances in the composite particles is not less than 50 wt % to not more than 90 wt %; and a weight-average dry particle size (Dw) of substantially spherical multinuclear particles formed of the magnetic substances is 20 nm or less.

Moreover, the present invention provides a dispersion solution prepared by dispersing composite particles in water or an aqueous solution, characterized in that the dispersion solution has a single-peak particle size distribution and a dispersity index (Dhw/Dhn) calculated from a number-average hydrodynamic particle size (Dhn) and a weight-average hydrodynamic particle size (Dhw) is 1.2 or less; the composite particles each have a structure in which substantially spherical multinuclear particles formed of a plurality of magnetic substances are surrounded by a film-state polymer compound; a polydispersity index (Dw/Dn) calculated from a number-average dry particle size (Dn) and a weight-average dry particle size (Dw) of the composite particles is 1.2 or less; the weight-average dry particle size (Dw) of the composite particles falls within the range of 50 nm to 300 nm; the content of the magnetic substances in the composite particles is not less than 50 wt % to not more than 90 wt %; and the weight-average dry particle size (Dw) of substantially spherical multinuclear particles formed of the magnetic substances is 20 nm or less.

The present invention can provide composite particles small in particle size, excellent in mono-dispersibility, high in content of a magnetic substance, large in saturation magnetization per particle, excellent in dispersion stability and having non-specific adsorption suppressibility, and a method for producing the same.

Furthermore, the present invention can provide a dispersion solution using the composite particles mentioned above.

Moreover, the present invention can provide a magnetic biosensing apparatus using the composite particles mentioned above and a magnetic biosensing method.

The present invention can provide composite particles applicable to a wide variety of industrial fields including medical materials, particularly, magnetic particles suitable for a magnetic biosensor, which magnetically detects the presence/absence and concentration of a target substance in a test solution, and can provide a method for producing the same.

Further features of the present invention will become apparent from the following description of exemplary embodiments with reference to the attached drawings.

### BRIEF DESCRIPTION OF THE DRAWINGS

FIG. 1A illustrates the steps of a method (an embodiment) for producing composite particles according to the present invention.

FIG. 1B is a schematic view of a composite particle (an embodiment) of the present invention.

FIG. 2 is a schematic view of a TMR sensor for use in the magnetic biosensing method of the present invention.

FIGS. 3A and 3B are schematic views illustrating a magnetic field formed by a composite particle placed on a magnetic sensor.

FIG. 4 illustrates a magnetic field distribution formed by composite particle B of the present invention on a magnetic sensor.

FIG. 5 is a transmission electron micrograph of magnetic particles obtained in Example 1.

FIG. 6A is a transmission electron micrograph of magnetic particles obtained in Example 9.

FIG. 6B is a transmission electron micrograph of FIG. 6A enhanced contrast by processing the image.

FIG. 7 is a curve chart illustrating a change of magnetic resistance of the TMR sensor used in Example 19.

FIG. 8A is a transmission electron micrograph showing fixation of composite particle B of Example 19 and an output of the corresponding TMR sensor.

FIG. 8B is a transmission electron micrograph showing fixation of composite particle B of Example 19 and an output of the corresponding TMR sensor.

FIG. 8C is a graph showing fixation of composite particle B of Example 19 and an output of the corresponding TMR sensor.

FIG. 9 is a schematic view illustrating fixation of composite particle B of Example 20.

### BEST MODE FOR CARRYING OUT THE INVENTION

The present invention will be described more specifically below.

A method for producing composite particles according to the present invention has

(1) mixing a first liquid and particles to prepare a mixture solution;

(2) mixing the mixture solution and a second liquid to prepare an emulsion containing a dispersoid formed of the first liquid and the particles;

(3) mixing a polymer compound with the emulsion; and

(4) fractionating the emulsion to extract the first liquid from the dispersoid to produce the composite particles each containing the particles and the polymer compound, characterized in that the dispersoid has a single-peak particle size distribution and a dispersity index (Dhw/Dhn) calculated from a number-average hydrodynamic particle size (Dhn) and a weight-average hydrodynamic particle size (Dhw) is 1.5 or less.

The emulsion can be a mini-emulsion.

A dispersant can be contained in the second liquid.

The first liquid can be an organic solvent insoluble in the second liquid or a monomer.

The second liquid can be water or an aqueous solution.

The polymer compound can be varied between an insoluble state and a soluble state depending upon the pH of the second liquid.

In addition to the aforementioned steps, changing the pH of the emulsion from the pH at which the polymer compound is soluble in the second liquid to the pH at which the polymer compound is insoluble can be included.

The polymer compound can be an amphipathic polymer compound having a hydrophobic site and a hydrophilic site.

The polymer compound can have a carboxyl group.

The polymer compound can have an amino group.

The particles can be particles containing an inorganic material.

The inorganic material can be a magnetic substance.

The weight-average dry particle size (Dw) of the magnetic substance can be 20 nm or less.

The magnetic substance can be a ferromagnetic metal, an alloy or oxide containing at least one ferromagnetic metal.

The ferromagnetic metal, alloy or oxide containing at least one ferromagnetic metal can be ferrite.

The ferromagnetic metal, alloy or oxide containing at least one ferromagnetic metal can be platinum-iron.

In addition to the aforementioned steps, allowing the polymer compound to adsorb an affinity ligand can be added.

In addition to the aforementioned steps,

(5) allowing the composite particles to adsorb a polymerization initiating group; and

(6) starting polymerization of monomers from the polymerization initiating group to obtain a polymer of monomers, can be added.

The polymerization initiating group can be a radical polymerization initiating group.

The polymerization initiating group can be a living radical polymerization initiating group.

The living radical polymerization initiating group can be a light iniferter polymerization initiating group.

The living radical polymerization initiating group can be an atom-transfer radical polymerization initiating group.

The polymer of monomers can have hydrophilicity.

The polymer of monomers can at least partly contain a functional group having non-specific adsorption suppressibility.

The polymer of monomers can at least partly contain a carboxyl group.

The polymer of monomers can at least partly contain a carboxy-betaine structure represented by the general formula (1) below.

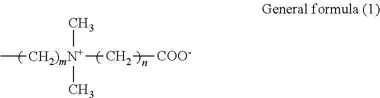

(where m and n represent an integer of 1 to 10)

In addition to the aforementioned steps,

(7) allowing the polymer of monomers to adsorb an affinity ligand can be added.

Furthermore, composite particles according to the present invention are the composite particles having a structure in which substantially spherical multinuclear particles each formed of a plurality of magnetic substances are surrounded by a film-state polymer compound, characterized in that a polydispersity index (Dw/Dn) calculated from a number-average dry particle size (Dn) and a weight-average dry particle size (Dw) of the composite particles is 1.2 or less; the content of the magnetic substances in the composite particles is not less than 50 wt % to not more than 90 wt %; and the weight-average dry particle size (Dw) of substantially spherical multinuclear particles formed of the magnetic substances is 20 nm or less.

The composite particles can at least partly have a hollow structure.

The weight-average dry particle size (Dw) of the composite particles can fall within the range of 50 nm to 300 nm.

The polymer compound can have a carboxyl group.

The polymer compound can have an amino group.

The magnetic substance can be a ferromagnetic metal, an alloy or oxide containing at least one ferromagnetic metal.

The ferromagnetic metal, alloy or oxide containing at least one ferromagnetic metal can be ferrite.

The ferromagnetic metal, alloy or oxide containing at least one ferromagnetic metal can be platinum-iron.

The polymer compound can adsorb an affinity ligand.

The composite particles can absorb the polymer of monomers having non-specific adsorption suppressibility around the particles.

The polymer of monomers can have hydrophilicity.

The polymer of monomers can at least partly contain a functional group having non-specific adsorption suppressibility.

The polymer of monomers can at least partly contain a carboxyl group.

The polymer of monomers can at least partly contain a carboxy-betaine structure represented by the general formula (1) above.

The polymer of monomers can adsorb an affinity ligand.

Furthermore, a dispersion solution according to the present invention is a dispersion solution formed by dispersing the composite particles in water or an aqueous solution, characterized in that the dispersion solution has a single-peak particle size distribution and a dispersity index (Dhw/Dhn) calculated from a number-average hydrodynamic particle size (Dhn) and a weight-average hydrodynamic particle size (Dhw) is 1.2 or less; the composite particles have a structure in which substantially spherical multinuclear particles each formed of a plurality of magnetic substances are surrounded by a film-state polymer compound; a polydispersity index (Dw/Dn) calculated from a number-average dry particle size (Dn) and a weight-average dry particle size (Dw) of the composite particles is 1.2 or less; the content of the magnetic substances in the composite particles is not less than 50 wt % to not more than 90 wt %; and the weight-average dry particle size (Dw) of substantially spherical multinuclear particles formed of the magnetic substances is 20 nm or less.

The composite particles can at least partly have a hollow structure.

The weight-average dry particle size of the composite particles can fall within the range of 50 nm to 300 nm.

The polymer compound can have a carboxyl group.

The polymer compound can have an amino group.

The magnetic substance can be a ferromagnetic metal, an alloy or oxide containing at least one ferromagnetic metal.

The ferromagnetic metal, alloy or oxide containing at least one ferromagnetic metal can be ferrite.

The ferromagnetic metal, alloy or oxide containing at least one ferromagnetic metal can be platinum-iron.

The polymer compound can adsorb an affinity ligand.

The polymer of monomers having non-specific adsorption suppressibility can adsorb around the composite particles.

The polymer of monomers can have hydrophilicity.

The polymer of monomers can at least partly contain a functional group having non-specific adsorption suppressibility.

The polymer of monomers can at least partly contain a carboxyl group. The polymer of monomers can at least partly contain a carboxy-betaine structure represented by the general formula (1) above.

The polymer of monomers can adsorb an affinity ligand.

A magnetic biosensing apparatus according to the present invention is characterized by having the above composite particles A and a magnetic sensor.

Furthermore, a magnetic biosensing method according to the present invention is characterized by including binding a target substance trapping substance to the surface of the composite particles A to obtain composite particles B capable of trapping the target substance; bringing the composite particles B capable of trapping the target substance into contact with a sample to trap the target substance in the sample; and detecting the composite particles B trapping the target substance by the magnetic sensor to determine the presence/absence or concentration of the target substance in the sample.

Fixing the composite particles B trapping the target substance to the surface of the magnetic sensor and applying a static magnetic field to the composite particles B fixed on the surface of the magnetic sensor can be included.

Next, a method for producing composite particles according to the present invention will be described.

The method for producing composite particles of the present invention includes

(1) mixing a first liquid and particles to prepare a mixture solution;

(2) mixing the mixture solution and a second liquid to prepare an emulsion containing a dispersoid formed of the first liquid and the particles;

(3) mixing a polymer compound with the emulsion; and

(4) fractionating the emulsion to extract the first liquid from the dispersoid to produce the composite particles each containing the particles and the polymer compound,

characterized in that the dispersoid has a single-peak particle size distribution and a dispersity index (Dhw/Dhn) calculated from a number-average hydrodynamic particle size (Dhn) and a weight-average hydrodynamic particle size (Dhw) is 1.5 or less.

As the first liquid and the second liquid in the present invention, a combination of liquids substantially not mixed with each other is selected. The first liquid can be an organic solvent and the second liquid can be water or an aqueous solution. Examples of such an organic solvent may include hydrocarbon solvents (such as hexane, heptane and octane), aromatic hydrocarbon solvents (such as benzene, toluene and xylene), halogenated hydrocarbon solvents (such as dichloromethane, chloroform, chloroethane and dichloroethane), ether solvents (such as ethyl ether, diethyl ether and isobutyl ether), ester solvents (such as ethyl acetate and butyl acetate) and ketone solvents (such as methyl ethyl ketone and methyl isobutyl ketone). Particularly, a hydrocarbon solvent, an aromatic hydrocarbon solvent and a halogenated hydrocarbon solvent are suitable. However, the organic solvent of the present invention is not limited to these. These organic solvents may be used alone or as a mixture of a plurality of types as long as the object of the present invention can be attained.

As the first liquid, a monomer can be also used.

The monomer is a compound insoluble in water or an aqueous solution and having a polymerizable ethylenyl unsaturated bond. As specific examples of the monomer that can be used, for example, in radical polymerization, following compounds may be mentioned.

That is, polymerizable unsaturated aromatic compounds such as styrene, chlorostyrene, α-methylstyrene, divinylbenzene and vinyltoluene;

polymerizable unsaturated carboxylic acids such as (meth)acrylic acid, itaconic acid, maleic acid and fumaric acid; polymerizable unsaturated sulfonic acids such as sodium styrene sulfonate or salts thereof;

polymerizable carboxylic esters such as methyl(meth)acrylate, ethyl(meth)acrylate, n-butyl(meth)acrylate, 2-hydroxyethyl(meth)acrylate, glycidyl(meth)acrylate, ethylene glycol di(meth)acrylate and tribromophenyl(meth)acrylate;

unsaturated carboxylic acid amides such as (meth)acrylonitrile, (meth)acrolein, (meth)acrylic amide, methylene bis(meth)acrylic amide, butadiene, isoprene, vinyl acetate, vinyl pyridine, N-vinyl pyrrolidone, vinyl chloride, vinylidene chloride and vinyl bromide;

polymerizable unsaturated nitriles, vinyl halides and conjugated dienes; and

macromonomers having a polymer segment such as polystyrene, polyethylene glycol or polymethyl methacrylate and a polymerizable functional group such as a vinyl group, a methacryloyl group or a dihydroxyl group.

As specific examples of the monomer to be used addition polymerization, the following compounds may be mentioned. That is, aliphatic or aromatic isocyanates such as diphenylmethane diisocyanate, naphthalene diisocyanate, tolylene diisocyanate, tetramethylxylene diisocyanate, xylene diisocyanate, dicyclohexane diisocyanate, dicyclohexylmethane diisocyanate, hexamethylene diisocyanate and isophorone diisocyanate; ketenes, epoxy group-containing compounds and vinyl group-containing compounds. As the monomer to be reacted with the above compounds, compounds having a functional group with activated hydrogen, such as a hydroxy group or an amino group may be mentioned. Specific examples thereof may include polyols such as ethylene glycol, diethylene glycol, propylene glycol, 1,4-butanediol, 1,6-hexanediol, glycerin, trimethylol propane, pentaerythritol, sorbitol, methylene glycoside, sucrose and bis(hydroxyethyl)benzene;

polyamines such as ethylenediamine, hexamethylenediamine, N,N′-diisopropylmethylenediamine, N,N′-di-sec-butyl-p-phenylenediamine and 1,3,5-triaminobenzene; and oximes.

In the present invention, the monomers may be used alone or in a combination of two or more types.

Furthermore, other than monomers, a polyfunctional compound that may serve as a crosslinking agent, may be used in combination. As the polyfunctional compound, the followings may be mentioned. That is, N-methylol acrylamide, N-ethanol acrylamide, N-propanol acrylamide, N-methylol maleimide, N-ethylol maleimide, N-methylol maleamic acid, N-methylol maleamic acid ester, N-alkylolamide of a vinyl aromatic acid (e.g., N-methylol-p-vinylbenzamide) and N-(isobutoxymethyl)acrylic amide. Moreover, of the aforementioned monomers, polyfunctional monomers such as divinylbenzene, divinylnaphthalene, divinylcyclohexane, 1,3-dipropynylbenzene, ethylene glycol di(meth)acrylate, diethylene glycol di(meth)acrylate, butylene glycol, trimethylol ethane tri(meth)acrylate and pentaerythritol tetra(meth)acrylate can be used as a crosslinking agent. Note that the polyfunctional compounds may be used alone or as a mixture of two types or more.

As the first liquid in the present invention, an organic solvent and a monomer may be used as a mixture.

When a monomer is used as the first liquid of the present invention, the first liquid is fractionated from the dispersoid of a mini-emulsion and thereafter the monomer can be polymerized.

As examples of the polymerization initiator that can be used in the present invention, the following initiators may be mentioned. That is, azo(azobisnitrile) based initiators such as
2,2′-azobis isobutyronitrile,
2,2′-azobis-(2-methylpropanenitrile), 2,2′-azobis-(2,4-dimethylpentanenitrile),
2,2′-azobis-(2-methylbutane nitrile), 1,1′-azobis-(cyclohexanecarbonitrile), 2,2′-azobis-(2,4-dimethyl-4-methoxyvarelonitrile), 2,2′-azobis-(2,4-dimethylvarelonitrile) and 2,2′-azobis-(2-amidinopropane)hydrochloride; and

peroxide based initiators such as benzoyl peroxide, cumene hydroperoxide, hydrogen peroxide, acetyl peroxide, lauroyl peroxide, a persulfate (e.g., ammonium persulfate), a peracid ester

(e.g., t-butyl peroctoate, α-cumylperoxypivalate and t-butylperoctoate).

In addition, mention may be made of ascorbic acid/iron sulfate (II)/sodium peroxyldisulfate, tert-butyl hydroperoxide/sodium sulfite, and tert-butyl hydroperoxide/sodium hydroxymethane sulfite.

Note that a redox initiator, whose components, for example, a reducing component is a mixture, such as a mixture of a sodium salt such as hydroxymethane sulfite and sodium sulfite, may be used.

The polymerization initiators can be used alone or in a combination of a plurality of types.

Each of these polymerization initiators may be added to the first liquid or the second liquid before emulsification or after emulsification. When added after emulsification, the polymerization initiator may be added either before or after fractionation.

On the other hand, a dispersant can be added to the second liquid, as needed. The dispersant is not particularly limited as long as the present invention can be attained; however, a low-molecular weight dispersant can be used in order to form a good emulsion. In the present invention, a dispersant having a weight-average molecular weight of 1000 or less is referred to as a low-molecular weight dispersant for convenience sake. When a dispersant having a weight-average molecular weight larger than 1000 is used, the viscosity of the second liquid becomes too large to obtain a good emulsion.

Examples of the dispersant may include an anionic dispersant, a cationic dispersant and a nonionic dispersant.

Examples of the anionic dispersant may include the following compounds: i.e., dodecylbenzene sulfonate, decyl benzene sulfonate, undecylbenzene sulfonate, tridecylbenzene sulfonate and nonylbenzene sulfonate; and sodium, potassium and ammonium salts of these.

Examples of the cationic dispersant may include the following compounds: i.e., cetyl trimethyl ammonium bromide, hexadecyl pyridinium chloride and hexadecyl trimethyl ammonium chloride.

Examples of the nonionic dispersant may include polyvinyl alcohol and commercially available dispersants. Of the dispersants mentioned above, an anionic dispersant is particularly suitable.

Depending upon the purpose, a polymerizable dispersant can be used as the dispersant. The polymerizable dispersant is defined as a dispersant having a reactive group polymerizable with a monomer. Examples of the reactive group may include unsaturated ethylene groups such as a vinyl group, an allyl group and a (meth)acryloyl group. Specific examples of the reactive dispersant are described in Japanese Patent Application Laid-Open No. H09-279073.

As long as the object of the present invention can be attained, the dispersants may be used alone or as a mixture of two types or more. The use amount of a dispersant is not particularly limited. As the use amount of a dispersant increases, the particle size of the mini-emulsion decreases. Accordingly, the particle size of composite particles tends to decrease.

The particles to be used in the present invention are particles containing an inorganic material such as a metal material, an oxide material or a semiconductor material, and can be appropriately selected depending upon the desired composite particles.

Particularly, when the production method of the present invention is employed as a method for producing magnetic particles, the particles may be used as a magnetic substance. The magnetic substance may be arbitrarily selected depending upon the purpose. However, a magnetic substance having a residual magnetization (which is a magnetization remaining after a magnetic field as strong as 5000 oersted is applied to a magnetic substance and the magnetic field is removed) is ⅓ as low as saturation magnetization, which is a magnetization at the time a magnetic field of 5000 oersted is applied can be used.

As the magnetic substance mentioned above, mention may be made of ferromagnetic metals such as iron, manganese, cobalt, nickel and gadolinium, or alloys or oxides containing at least one of them. Specific examples thereof may include magnet steel containing iron as a main component and cobalt, tungsten, chrome, nickel and aluminum as an additive; alloys of iron with platinum and/or neodymium and/or samarium, an alloy of samarium and cobalt and ferrites such as triiron tetraoxide (Fe₃O₄), CoFe₂O₄, and γ-sesquioxide (γ-Fe₂O₃).

For use in a magnetic biosensor, magnetic particles having a saturation magnetization tend to be required. Therefore, of these metals, the metals classified into a ferromagnetic substance, in a bulk state, can be used. In addition, a magnetic substance formed of magnetic particles is to be dispersed in an aqueous solution such as body fluid and the surface thereof is to be modified with an antibody. For the reasons, the metals having poor reactivity can be used. Furthermore, since the composite particles of the present invention are produced by use of an emulsion, metals that can easily form particles are preferable. In view of these, ferrites are more preferable, and particularly, Fe₃O₄ (magnetite) is preferable. Furthermore, from the same viewpoint, an Fe alloy is preferable and particularly, platinum-iron is preferable. However, the type of magnetic substance is not limited as long as the object of the present invention can be attained.

Note that, in the present invention, depending upon the purpose, a surface-modified magnetic substance can be used, which is treated with various type of coupling agent represented by a silane coupling agent or with a known surface treatment agent such as a higher fatty acid. As the purpose of the surface treatment, for example, hydrophobic treatment and hydrophilic treatment may be mentioned.

The weight-average dry particle size (Dw) of particles is not particularly defined as long as it is smaller than Dw of desired composite particles. On the other hand, when the particles are used as a magnetic substance, if Dw is too small, large saturation magnetization cannot be obtained due to thermal fluctuation. Therefore, a magnetic substance generally having a Dw of 2 nm or more, preferably 5 nm or more, and more preferably 6 nm or more is used. However, Dw is preferably not more than 20 nm. The case where Dw is larger than 20 nm is not preferable, because the effect of the stray magnetic field increases with the result that a good mini-emulsion cannot be formed.

The emulsion in the present invention is characterized in that the emulsion has a single-peak particle size distribution and a dispersity index (Dhw/Dhn) calculated from a number-average hydrodynamic particle size (Dhn) and a weight-average hydrodynamic particle size (Dhw) is 1.5 or less. Since the mono-dispersibility of the emulsion contributes to the mono-dispersibility of the resultant composite particles, Dhw/Dhn is more preferably 1.2 or less.

The emulsion in the present invention may be a mini-emulsion. The mini-emulsion in the present invention is a mono-dispersion emulsion having a single-peak particle size distribution, the weight-average hydrodynamic particle size (Dhw) thereof falls within the submicron range (50 nm to 1000 nm), and a polydispersity index (Dhw/Dhn) calculated from a number-average hydrodynamic particle size (Dhn) and a weight-average hydrodynamic particle size (Dhw) is 1.5 or less. Since the mono-dispersibility of the mini-emulsion contributes to the mono-dispersibility of the resultant composite particles, a mono-dispersion emulsion more preferably has a Dhw of 50 nm to 500 nm and a Dhw/Dhn of 1.2 or less. The case where the emulsion of the present invention is a mini-emulsion is preferable since fractionation (described later) can be easily performed.

In the present invention, in order to stabilize the emulsion, hydrophobe (cosurfactant), which is soluble in the first liquid and has a solubility to the second liquid of 0.01 g/L or less, may be contained in the first liquid.

As specific examples of the hydrophobe to be used in the present invention, the followings may be mentioned:

(a) C8 to C30 straight, branched and cyclic alkanes such as hexadecane, squalane and cyclooctane;

(b) C8 to C30 alkyl acrylates such as stearyl methacrylate and dodecyl methacrylate;

(c) C8 to C30 alkyl alcohols such as cetyl alcohol;

(d) C8 to C30 alkyl thiol such as dodecyl mercaptan;

(e) polymers such as polyurethane, polyester and polystyrene; and

(f) long-chain aliphatic or aromatic carboxylic acids, long chain aliphatic or aromatic carboxylic acid esters, long-chain aliphatic or aromatic amines, ketones, halogenated alkanes, silanes, siloxanes and isocyanates.

Long-chain oil soluble initiators such as lauroyl peroxide can be used. Of them, an alkane having 12 carbon atoms or more is preferable and an alkane having 12 to 20 carbon atoms is more preferable.

The emulsion of the present invention can be prepared by a known emulsification method in the art.

Examples of the known method in the art may include an intermittently shaking method, a stirring method using a mixer such as a propeller-form mixer and a turbine type mixer, a colloid-mill method, a homogenizing method and an ultrasonic irradiation method. As a method for obtaining a relatively large size mono-dispersion emulsion, mention may be made of a method using a micro-reactor such as a membrane emulsion method using an SPG membrane, a micro-channel emulsification method and a branched micro-channel emulsification method. These methods can be used alone or in a combination with a plurality of types. Furthermore, the mini-emulsion of the present invention may be prepared in a single-step emulsification or in a multiple-step emulsification.

The polymer compound to be used in the present invention is characterized by presenting an insoluble state or a soluble state depending upon the pH (difference) of the second liquid. Particularly, in order to interact with dispersoid of the emulsion, improve dispersion stability of the dispersoid and prevent dissociation of the particles from the dispersoid, an amphoteric polymer compound having a hydrophobic portion and a hydrophilic portion is suitable.

As examples of the hydrophobic portion of the amphoteric polymer compound, mention may be made of polymers or copolymers including styrene, a styrene derivative such as α-methylstyrene, vinyl cyclohexane, a vinyl naphthalene derivative, an acrylic ester and an methacrylic ester. The hydrophobic portion is not limited to these as long as the object of the present invention can be attained.

As examples of the hydrophilic portion of the amphoteric polymer compound, polymers or copolymers containing a site having a functional group changing a degree of dissociation in response to pH can be used. In the amphoteric polymer compounds containing such a site, in which the functional group is dissociated, the affinity for water or an aqueous solution improves. In the case where the functional group is not dissociated, the affinity for water or an aqueous solution decreases. As a result, the solubility thereof to water or an aqueous solution changes depending upon pH (change). Examples of such a functional group may include a carboxyl group and an amino group. However, the functional group is not limited to these as long as the purpose of the present invention can be attained.

In the present invention, the solubility of a polymer compound can be evaluated by the solubility test that will be described below.

A polymer compound is mixed to the second liquid controlled at an arbitral pH so as to obtain a concentration of 2 wt % and shaken at 25° C. for 24 hours and allowed to stand still for 24 hours. Then, the light (550 nm) transmitted through the second solution containing the polymer compound is evaluated. The case where a transmissivity is 99%, the polymer is determined as soluble. The case where a transmissivity is less than 99%, the polymer is determined as insoluble. As an apparatus for evaluating a transmissivity, for example, U-2001 type double-beam spectrophotometer (Hitachi Ltd.) is known.

The weight-average molecular weight of the polymer compound in the present invention is not less than 500 to not more than 1000000, and preferably not less than 1000 to not more than 100000. The case where the weight-average molecular weight exceeds 1000000 is not preferable because a degree of intramolecular and intermolecular tangling becomes large, increasing the viscosity of the emulsion. On the other hand, the case where the weight-average molecular weight is less than 500 is not preferable, because the effect of improving the dispersion stability of the emulsion and the effect of preventing dissociation of the particles from the dispersoid decrease.

The weight-average molecular weight can be measured by a light scattering method, an X-ray micronucleus dispersion method, a sedimentation equilibrium method, a diffusion method, an ultra-centrifugation method, and various chromatographic methods. In the present invention, the weight-average molecular weight is a weight-average molecular weight in terms of polystyrene measured by GPC (gel permeation chromatography).

The fractionation employed in the present invention is an operation of preferentially extracting a target liquid component from the dispersoid of an emulsion. For example, when the dispersoid is formed of particles and an organic solvent, the organic solvent alone is extracted from the dispersoid by fractionation. When a liquid component is formed of a plurality of organic solvents and monomers, one of the organic solvents can be preferentially extracted by use of, e.g., different boiling points of the components. Furthermore, in the fractionation of the present invention, an extraction degree of a liquid component can be appropriately varied depending upon the purpose.

Fractionation can be carried out by any one of the known methods in the art. For example, mention may be made of fractionation performed under reduced pressure for preferentially fractionating a liquid component having a low boiling point by use of a pressure reducing apparatus such as an evaporator, fractionation for extracting a liquid component by adding a solvent compatible with a target liquid component to an emulsion, and fractionation for extracting a liquid component by use of dialysis. The fractionation performed under reduced pressure by use of a pressure reducing apparatus has an advantage of easily controlling the degree of fractionation by pressure reduction conditions. In the present invention, a plurality of fractionation methods may be used in combination.

An exemplary embodiment of a method for producing composite particles of the present invention include:

(1) mixing a first liquid and particles to prepare a mixture solution;

(2) mixing the mixture solution and a second liquid to prepare an emulsion;

(3) mixing a polymer compound with the emulsion; and

(4) fractionating the first liquid from the dispersoid of the emulsion, and include adhering the polymer compound to the dispersoid by changing pH of the emulsion from the pH at which the polymer compound is soluble to the second liquid to the pH at which the polymer compound is insoluble, characterized in that the emulsion has a single-peak particle size distribution and a dispersity index (Dhw/Dhn) calculated from a number-average hydrodynamic particle size (Dhn) and a weight-average hydrodynamic particle size (Dhw) is 1.5 or less.

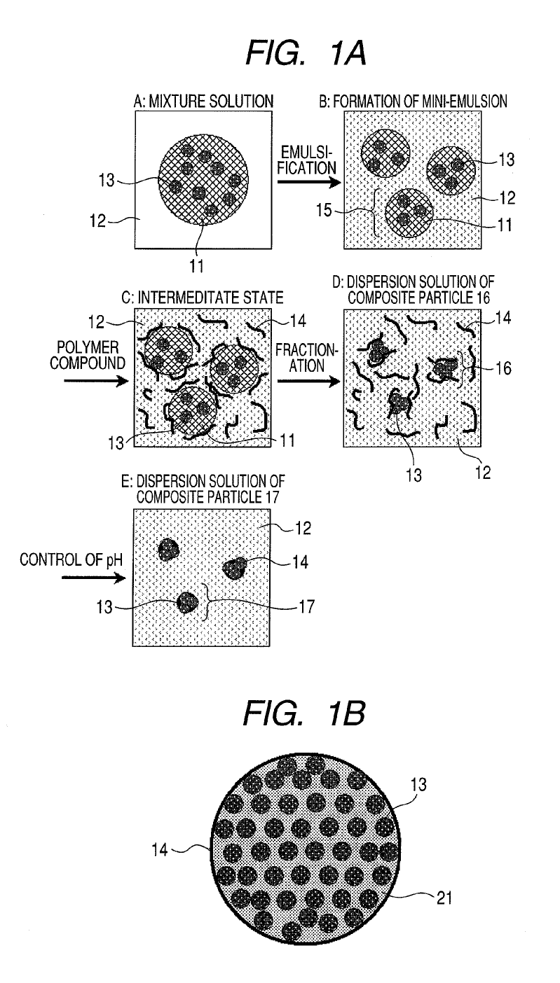

The exemplary embodiment of a method for producing composite particles of the present invention will be described based on FIG. 1A. FIG. 1A illustrates the steps of a method (an embodiment) for producing composite particles according to the present invention.

### (Step A: Mixture Solution)

Mixture solution A is prepared by dispersing particles 13 in a first liquid 11.

### (Step B: Formation of Emulsion)

Mixture solution A is mixed with a second liquid 12 and emulsified to form an emulsion. The emulsion formed herein is excellent in mono-dispersibility, and more preferably a mini-emulsion. A dispersoid is represented by reference numeral 15.

### (Step C: Intermediate State)

When a polymer compound 14 is mixed with emulsion B, the polymer compound 14 interacts with the dispersoid 15 of the emulsion and stabilizes the dispersion of the emulsion. Furthermore, the polymer compound 14 serves also as a blocking agent for preventing dissociation of the particles 13 from the dispersoid 15.

### (Step D: Dispersion Solution of Composite Particles 16)

Dispersion solution D of composite particles 16 will be described. The solution in the intermediate state C is fractionated to extract only the first liquid 11 from the dispersoid 15. As a result, the particles 13 come to aggregate by using the dispersoid 15 of the emulsion as a template. As a result, composite particles 16 having a highly uniform particle size are obtained. Since the polymer compound 14 is adsorbed onto the surface of the composite particles 16, the composite particles 16 exhibit dispersion stability.

### (Step E: Dispersion Solution of the Composite Particles 17)

Dispersion solution of the composite particles 17 will be described. The pH of Dispersion solution D of the composite particles 16 is controlled to insolubilize the polymer compound 14. The polymer compound 14 insolubilized precipitates on the surface of the composite particles 16 and deposited to form a thin film to obtain composite particles 17. The thickness of the thin film formed on the surface of the composite particles 17 can be controlled by controlling the degree of insolubility of the polymer compound 14 by pH control and changing operation time.

Next, the composite particles in the present invention will be described based on the FIG. 1B. FIG. 1B is a schematic view of a composite particle according to an embodiment of the present invention.

The composite particle of the present invention is a composite particle having a structure where spherical multinuclear particles each formed of a plurality of particles 13 (magnetic substances herein) are surrounded by a film-state polymer compound 14. The portion except the solid microparticles 13 constituting multinuclear particles is a matrix member 21. The matrix member 21 is a polymer of monomers previously described or a surface modifier of the particles 13 or both of them.

When the composite particles are the magnetic particles to be used in a magnetic biosensor, the mono-dispersibility is extremely important. If the mono-dispersibility is not sufficient, reproducible quantification of a target substance by the magnetic biosensor cannot be performed. Therefore, a polydispersity index (Dw/Dn) calculated from Dw and a number-average dry particle size (Dn) of the composite particles is 1.2 or less and further preferably 1.1 or less.

To reproduce sufficient detection sensitivity of a magnetic biosensor, saturation magnetization per composite particle must be sufficiently large. The magnitude of saturation magnetization varies depends upon the content of the particles 13 in a composite particles. The content is not less than 50 wt % and less than 90 wt %. When the content is less than 50 wt %, sufficient saturation magnetization cannot be obtained. When the content is 90 wt % or more, composite particles collapse and the particles 13 are exposed on the surface of composite particles. As a result, the dispersibility may decrease.

Furthermore, when the particles 13 contained in composite particles of the present invention are formed of a magnetic substance, Dw of the particles 13 is characterized by being 20 nm or less. When Dw is larger than 20 nm, the particles 13 may be exposed on the surface of composite particles. As a result, the dispersibility may decrease.

Dw of the composite particles are not particularly limited. When the composite particles are used as magnetic particles for use in a magnetic biosensor, Dw is preferably not less than 50 nm and less than 300 nm. When Dw is smaller than 50 nm, the saturation magnetization per composite particle cannot be maintained sufficiently large. As a result, it is difficult to reproduce sufficient detection sensitivity. On the other hand, when Dw is 300 nm or more, the mobility of the composite particles decreases. As a result, the detection speed may significantly decrease.

The composite particle has a structure in which substantially spherical multinuclear particles formed of a plurality of magnetic substances are surrounded by a film-state polymer compound.

The composite particles of the present invention preferably have an average aspect ratio (major axis/minor axis) within the range of 1.0 to 1.5, and more preferably in the range of 1.0 to 1.2, in short, preferably have enhanced sphericity. Such true spherical composite particles are advantageous because good flowability is shown when they are used in being dispersed in a liquid.

Whether a thin film of the polymer compound 14 is formed on the surface of the composite particles or not can be determined by a known measurement or calculation method in the art; however it is preferred to visually confirm it by a transmission electron microscope (TEM). However, when the thickness of the thin film is too thin to evaluate by TEM, determination can be made by use of an electrophoretic method and a surface analysis method based on surface element analysis in combination.

The composite particles of the present invention may have voids such as a hollow structure partly or wholly within the particles. When the composite particles have a hollow structure, the specific gravity of the composite particles decreases. As a result, sedimentation and the resultant aggregation may be suppressed in some cases.

As a method for producing composite particles having a hollow structure, a known method in the art is applicable. The hollow structure is presumably formed since a magnetic substance is condensed on the wall surface of oil drops (dispersoid) of an emulsion to aggregate during a fractionation process and the aggregation structure is frozen. Therefore, a fractionation rate is increased more than usual in view of convenience. Furthermore, the same effect can be obtained by reducing the content of magnetite in oil drops even at a general fractionation rate.

The content of particles 13 in a composite particle can be measured or calculated by a known method in the art; however, preferably measured based on thermogravimetry.

Furthermore, Dw and Dn of the composite particles and particles in the present invention can be measured or calculated by a known method in the art; however, they are preferably evaluated based on the value obtained by observing a dry state composite particle by a transmission electron microscope (TEM).

Next, the dispersion solution of the present invention will be described. The dispersion solution in the present invention is a dispersion solution prepared by dispersing the composite particles in water or an aqueous solution characterized in that the dispersion solution has a single-peak particle size distribution and a dispersity index (Dhw/Dhn) calculated from a number-average hydrodynamic particle size (Dhn) and a weight-average hydrodynamic particle size (Dhw) is 1.2 or less; and the composite particles have a structure in which substantially spherical multinuclear particles each formed of a plurality of magnetic substances are surrounded by a film-state polymer compound, a polydispersity index (Dw/Dn) calculated from a number-average dry particle size (Dn) and a weight-average dry particle size (Dw) of the composite particles is 1.2 or less; the content of the magnetic substances in the composite particles is not less than 50 wt % to not more than 90 wt %; and the weight-average dry particle size (Dw) of substantially spherical multinuclear particles formed of the magnetic substances is 20 nm or less.

When the dispersion solution is applied as a composition to be contained in a test solution for use in a magnetic biosensor, the dispersion stability is extremely important. Therefore, the dispersion solution has a single-peak particle size distribution. Furthermore, if a mono-dispersibility is not sufficient, reproducible quantification of a target substance by the magnetic biosensor cannot be performed. Preferably, Dhw/Dhn of the dispersion solution is 1.2 or less, and further preferably 1.1 or less.

The particle size distribution of the emulsion and the dispersion solution of the present invention and Dhn and Dhw can be measured by a particle-size measuring method known in the art; however, preferably measured based on a dynamic light scattering method. As a specific measuring device, a dynamic light scattering photometer DLS-8000 manufactured by Otsuka Electronics Co., Ltd. can be used.

In the present invention, composite particles may be obtained by mixing a first liquid and particles to prepare a mixture solution; mixing the mixture solution and a second liquid to prepare an emulsion; mixing a polymer compound with the emulsion; and fractionating the emulsion to extract the first liquid from the dispersoid; and adsorbing a polymerization initiating group to the composite particles; and polymerizing monomers from the polymerization initiating group to obtain a polymer of monomers.

The emulsion herein is characterized by having a single-peak particle size distribution and a dispersity index (Dhw/Dhn) calculated from a number-average hydrodynamic particle size (Dhn) and a weight-average hydrodynamic particle size (Dhw) is 1.5 or less.

As the polymerization initiating group in the present invention, a known polymerization initiating group in the art, such as a radical polymerization initiating group, a cation polymerization initiating group, an anion polymerization initiating group can be used. In view of simple polymerization, a radical polymerization initiating group can be suitably used. Examples of the radical polymerization initiating group may include a functional group containing a self-decomposable structure like an azo compound and a peroxide; and a functional group containing a structure generating an active species by adding a catalyst, etc., just like a combination of a functional group containing diol with Ce⁴⁻. Note that the polymerization initiating group in the present invention is not limited to these.

The present invention is particularly preferably carried out when the polymerization initiating group is a living radical polymerization initiating group. The polymer of monomers formed by living radical polymerization has a small molecular-weight distribution compared to the polymer of monomers formed by general radical polymerization. Therefore, the polymers of monomers having a uniform chain length can be grafted on the surface. Furthermore, in the polymerization reaction, since activated species are uniformly generated from the living radical polymerization initiating group, a polymer of monomers can be grafted at a high density compared to the case of using the conventional radical polymerization initiating group. Grafting a polymer of monomers a uniform chain length on the surface of particles at a high density is generally known to greatly contribute to improvement of non-specific adsorption suppressibility and improvement of dispersibility.

As the living radical polymerization initiating group of the present invention, for example, a light iniferter polymerization initiating group, an atom-transfer radical polymerization initiating group and a nitroxide-mediated polymerization initiating group may be mentioned. Of them, use of a nitroxide-mediated polymerization initiating group is not preferable since a polymerizable monomer species is limited. On the other hand, a light iniferter polymerization initiating group is preferably used because this polymerization initiating group has advantages: the range of a polymerizable monomer species is wide and polymerization is particularly simple. More preferably, an atom-transfer radical polymerization initiating group is used since this polymerization initiating group has high reaction controllability.

As the light iniferter polymerization initiating group, a dithiocarbamate compound as represented by the general formula (2) below can be used, which generates an activated species involved in a polymerization reaction by irradiation of UV rays. More specifically, particles, on the surface of which a dithiocarbamate compound, for example, a compound having N,N-diethyldithiocarbamate group, is adsorbed, are dispersed in a reaction solvent. Thereafter, monomers are added. The reaction mixture is irradiated with UV rays to graft a polymer of the monomers having a uniform chain length on the surface of the particles.

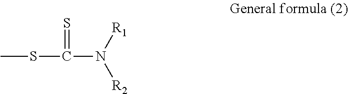

(where R₁ and R₂ represents an alkyl group, substituted alkyl group, aryl group or substituted aryl group having one or more carbon atoms.)

As the atom-transfer radical polymerization initiating group, a known group can be used. Primarily, functional groups contained by an organic halide and a halogenated sulfonyl compound, which have highly reactive carbon-halogen bonds, may be mentioned. In the atom-transfer radical polymerization, when a transition metal complex serves as a catalyst to these functional groups, an activated species capable of initiating living polymerization is generated. More specifically, particles having an atom-transfer radical polymerization initiating group, for example, an organic halide, adsorbed onto the surface are dispersed in a reaction solvent. Thereafter, a monomer and a transition metal complex serving as a catalyst are added and heated, if necessary, to graft a polymer of monomers having a uniform chain length on the surface of the particles.

As the transition metal complex, a complex formed of a halogenated metal and a ligand can be used. As examples of metal species of the halogenated metals, transition metals, for example, from Ti of atomic No. 22 to Zn of atomic No. 30 are preferable. Of them, Fe, Co, Ni and Cu are particularly preferable.

The use amount of transition metal complex is preferably not less than 0.0001% by mass to not more than 10% by mass based on the use amount of monomers constituting a polymer of monomers, and more preferably not less than 0.05% by mass to not more than 5% by mass.

The ligand is not particularly limited as long as it can be coordinated to a halogenated metal. For example, use can be made of 2,2′-bipyridyl, 4,4-di-(n-heptyl)-2,2′-bipyridyl, 2-(N-pentyliminomethyl)pyridine, (−)-sparteine, tris(2-dimethylaminoethyl)amine, ethylenediamine, dimethylglyoxime, 1,4,8,11-tetramethyl-1,4,8,11-tetraazacyclotetradecane, 1,10-phenanthrorin, N,N,N′,N″,N″-pentamethyldiethylenetriamine, and hexamethyl(2-aminoethyl)amine.

As the adsorption manner of a polymerization initiating group to composite particles, chemical adsorption and physical adsorption may be mentioned. The chemical adsorption refers to an adsorption manner via a covalent bond. The physical adsorption refers to an adsorption manner via the van der Waals force. Of them, the chemical adsorption is preferably used since it has stronger bonding force. Examples of the chemical adsorption may include amide bonding, ester bonding, ether bonding, thioether bonding, thioester bonding and urethane bonding. The present invention is not limited to these examples. Furthermore, a plurality of adsorption manners may be used in combination as long as the object of the present invention can be attained.

In the present invention, a polymer of monomers can be obtained by polymerizing monomers. As the monomer of the present invention, a monomer having high affinity for water can be used. Non-specific adsorption of e.g., a biomolecule to the polymer of monomers in an aqueous solution is mainly caused by hydrophobic interaction between a hydrophobic amino acid site contained in the biomolecule and the polymer of monomers. This means that if the polymer of monomers has hydrophilicity, the non-specific adsorption of a biomolecule to the polymer of monomers can be reduced.

As the monomer of the present invention, a monomer containing a functional group having non-specific adsorption suppressibility in the structure thereof is preferably used. Examples of the functional group having non-specific adsorption suppressibility may include a hydroxyl group, a methoxy group, an ethoxy group, a propoxy group, a 2-hydroxyethyl group, a 2-hydroxypropyl group, a 3-hydroxypropyl group, a 2-hydroxyisopropyl group a 2-hydroxybutyl group, a 3-hydroxybutyl group, 4-hydroxybutyl group, a carboxyl group, a sulfonyl group, a phosphonyl group, an amino group, a methylamino group, an ethylamino group, an isopropylamino group, an amide group, methylamide group, an ethylamide group, an isopropylamide group, a pyrrolidone group, an ethylene glycol group and a polymer thereof, a choline group, a phosphatidylcholine group and derivatives of these. Of them, monomers at least partly having a carboxyl group is more preferably used. Of them, a monomer having a carboxy-betaine structure as represented by the general formula (1) above and known to exhibit high non-specific adsorption suppressibility is more preferable. If a carboxyl group is actively esterified, it is possible to fix other substances such as an antibody recognizing a target substance and an enzyme to the esterified site.

In the present invention, polymerization of monomers may be performed in a reaction solution containing a free polymerization initiator not adsorbed to composite particles. The molecular weight and molecular-weight distribution of a free polymer produced from the free polymerization initiator can be presumed to be equivalent to the molecular weight and molecular-weight distribution of the polymer compound grafted to the composite particles 17. Furthermore, the molecular weight and molecular weight-distribution of a free polymer can be measured by GPC (trade name: AS-8020 manufactured by Tosho Corporation, eluant: water, standard polymer: polyethylene oxide).

As the free polymerization initiator, the same type of group as the polymerization initiating group can be selected. For example, in the case of particles to which an N,N-diethyldithiocarbamate group is introduced as a light iniferter polymerization initiating group, N,N-diethyldithiocarbamide acetic acid can be used as a free polymerization initiating species.

The free polymerization initiator is preferably added in a ratio of not less than 0.0001 mole equivalent to not more than 0.1 mole equivalent relative to the monomers constituting a polymer of monomers; and more preferably, not less than 0.0005 mole equivalent to not more than 0.05 mole equivalent.

The affinity ligand in the present invention is defined as a substance having affinity for a specific target substance, such as a physiologically active substance. Particularly, when the affinity particle of the present invention is used in a magnetic biosensor, the affinity ligand is a substance responsible for selecting a target substance in a test solution, for example, a substance (so called a receptor) selectively reacting directly to a target substance in a test solution and a substance involved in a reaction of a target substance (e.g., a substance selectively catalyzing a reaction of a target substance), etc. This trapping member may also have a function involved in displaying the presence/absence of detection and a detection level, more specifically, a function of emitting color by reacting with a substance released from a receptor and a residual substance. The trapping member to be used in the present invention may include, but not limited to, an enzyme, a sugar chain, a catalyst, an antibody, an antibody fragment, an antigen and a nucleic acid.

On the other hand, the target substance in a test solution is a substance serving as a detection target, for example, a substance selectively binding to the trapping member, a substance selectively reacting directly with the trapping member and a substance involved in a reaction of the trapping member (e.g., substance selectively catalyzing the reaction of the trapping member). The target substance is not limited to a biological substance and the size thereof is not limited. Note that when a target substance is a biological substance contained in living organisms, such as a sugar, a protein, an amino acid, an antibody, an antigen and a pseudo-antigen, a vitamin and a nucleic acid, a relevant substance of these, a pseudo biological substance artificially synthesized, and optionally a fragment thereof, the object of the present invention can be satisfactorily attained.

In the present invention, as long as the binding capacity of an affinity ligand to a target substance is not inhibited, the position of a polymer compound or a polymer of monomers at which a trapping member is bound and a binding method are not particularly limited. For example, when the affinity ligand is a protein, as long as the carboxyl end and/or the amino end of the protein and the function of the affinity ligand are not inhibited, a polymer compound or a polymer of monomers can be bound at any position. As a method of binding the affinity ligand to a polymer compound or a polymer of monomers, a physical adsorption method and a chemical binding method, etc. may be mentioned.

The physical adsorption of an affinity ligand to a polymer compound or a polymer of monomers can be carried out by mixing the polymer compound or the polymer of monomers and the affinity ligand. In this way, the affinity ligand can be non-specifically adsorbed. This is preferred in view of operation simplicity.

On the other hand, as a method of binding an affinity ligand to a polymer compound or a polymer of monomers, chemical binding such as covalent binding can be used. The chemical binding is preferable compared to physical adsorption since the strong binding is obtained. As a method of covalently fixing an affinity ligand to a polymer compound or a polymer of monomers, if the affinity ligand is a protein, there is a method of reacting the amino group of an amino acid contained in the protein sequence and a carboxyl group attached as a charged functional group to the polymer compound according to a known method in the art.

Next, the magnetic biosensing apparatus and magnetic biosensing method of the present invention will be described.

The magnetic biosensing apparatus according to the present invention is characterized by having composite particles A mentioned above and a magnetic sensor.

Furthermore, the magnetic biosensing method according to the present invention is characterized by having binding a target-substance trapping agent to the surface of the composite particles A above to obtain composite particles B capable of trapping the target substance; bringing the composite particles B capable of trapping the target substance into contact with a sample to trap the target substance in the sample; and detecting the composite particles B trapping the target substance by the magnetic sensor to determine the presence/absence or concentration of the target substance in the sample.

Fixing the composite particles B trapping the target substance to the surface of the magnetic sensor and applying a static magnetic field to the composite particles B fixed on the surface of the magnetic sensor can be included.

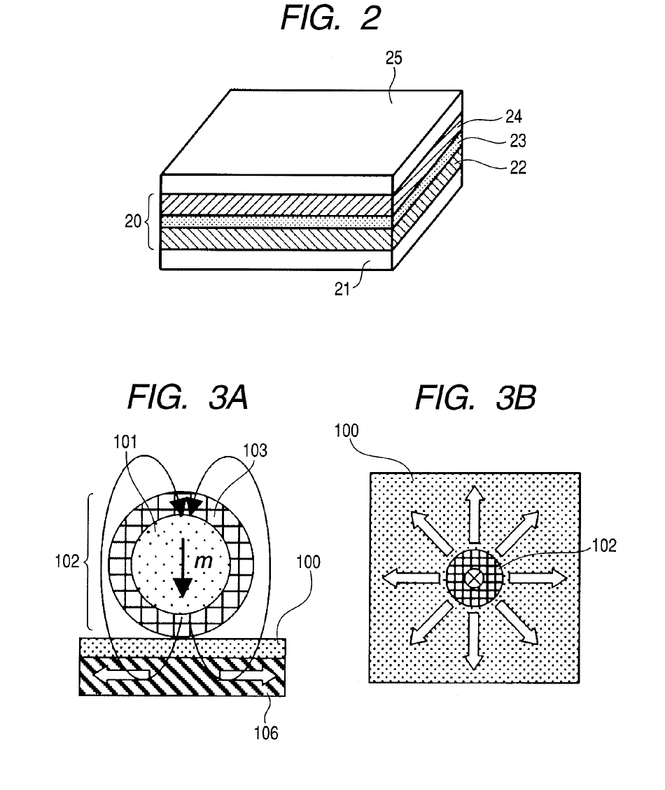

Next, how to detect the composite particles will be described with reference to FIGS. 2 to 4.

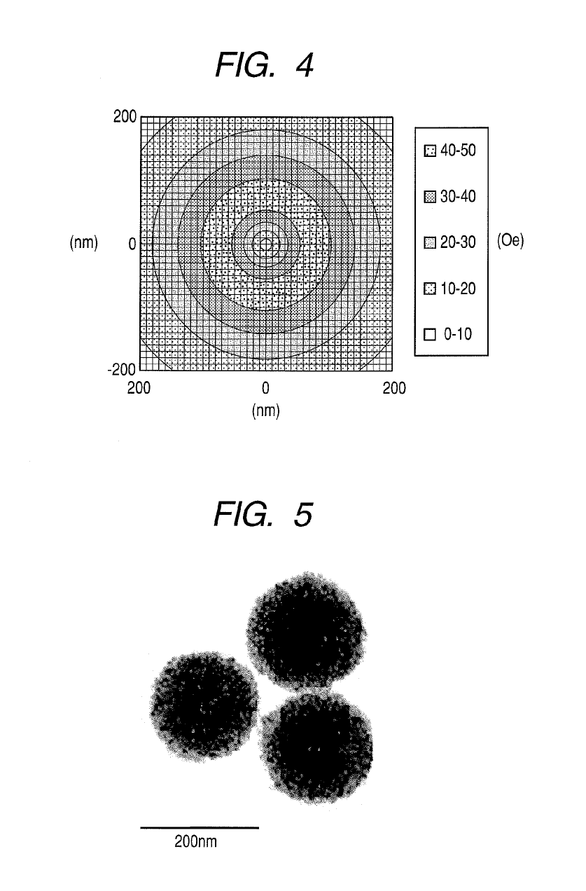

FIG. 2 is a schematic view of a TMR (tunnel magnetoresistance) sensor for use in the magnetic biosensing method of the present invention. FIGS. 3A and 3B are schematic views illustrating a magnetic field formed by a composite particle placed on a magnetic sensor. FIG. 4 illustrates a magnetic field distribution formed by composite particle B of the present invention on a magnetic sensor.

In the present invention, the presence/absence or concentration of a target substance fixed to the composite particles is determined by detecting the composite particles by a magnetic sensor.

### i) Magnetic Sensor

Any magnetic sensor can be used as long as it has a constitution capable of detecting the stray magnetic field from composite particle B102. For example, a magnetoresistance effect element, a hole element, a magnetoimpedance element and a flux gate element may be mentioned.

### ii) Fixation of Composite Particle on Magnetic Sensor

Composite particle B102, which is prepared by attaching a target substance trapping substance 103 to the surface of composite particle A101, and a target substance 104 are fixed onto the magnetic sensor. At this time, a material capable of trapping the target substance 104 can be prepared on the surface 100 of a detection region of the magnetic sensor. Furthermore, a material for preventing deposition of the target substance 104 and composite particles B102 can be also prepared on the surface of a non-detection region of the sensor.

iii) Detection of Composite Particle

The target substance fixed to composite particle B102 is detected by detecting the stray magnetic field from composite particle B102 fixed to the surface 100 of the magnetic sensor by the magnetic sensor. At this time, for example, a static magnetic field, which primarily consists of a component in the direction which has difficulty with detection of the sensor, is applied to composite particle B102. In this manner, larger stray magnetic field from composite particle B102 can be obtained without saturating the detectability of the magnetic sensor.

Note that as long as the stray magnetic field from composite particle B102 can be detected, any measurement method may be employed. Thus the measurement method is not limited to the aforementioned one. For example, (1) after a strong magnetic field Hₛ is externally applied to align the direction of magnetization of composite particle B102 parallel to a certain direction, Hₛ is reduced (or up to zero). In the conditions, residual magnetization or relaxation of magnetization of composite particle B102 may be observed. Alternatively, (2) as long as the strength of the magnetic field to be applied does not saturate detectability, an approach of using a bias magnetic field in the detectable direction of the magnetic sensor to be used may be employed.

Furthermore, when the target substance 104 is detected by detecting composite particle B102, the following constitution can be also employed. That is, a target substance trapping substances are prepared on the surface of composite particle B102 and the surface 100 of the magnetic sensor and the target substance 104 is sandwiched between the target substance trapping substances.

Now, detection of a target substance will be more specifically described by way of a TMR sensor; however, the magnetic sensor of the present invention is not limited to this.

First, as shown in FIG. 2, the TMR sensor to be used has a TMR element (TMR sensor) 20, which is formed of a first magnetic film 22, a second magnetic film 24 and a tunnel insulating film 23, an upper electrode 25 and a lower electrode 21, which are provided so as to sandwich the element. TMR sensor has a magnetic anisotropy in the element in-plane direction. The magnetic sensor of this type has features in that the magnetic field in the element in-plane direction can be detected; and that the electric resistance of the element to be measured through the upper and lower wiring varies depending upon the magnitude of the magnetic field.

Next, the case where single composite particle A101 having magnetization m is fixed onto the TMR sensor surface 100 will be discussed.

At this point, composite particle A101 according to the present invention having an average particle size of e.g., 175 nm has the following magnetization curve. Magnetization of composite particle A101 stands up sharply in the region of an external magnetic-field application value of 0 to 1 kOe and is virtually saturated at about 7 kOe. The magnitude of saturation magnetization is about 2.5×10⁻¹³ [emu/bead].

Next, when the direction of magnetization of composite particle A101 is perpendicular to the film surface of the magnetization sensing portion of the TMR sensor, the magnetic field received by the TMR sensor 106 from composite particle A101 will be discussed (FIG. 3A is a sectional view and FIG. 3B is a plan view). An open arrow of FIG. 3A indicates an in-plane component of the stray magnetic field Hₛ received by a magnetization sensing portion 106 of the TMR sensor from the composite particle. A direct-current magnetization is applied (not shown) in the direction perpendicular to the film surface of the magnetization sensing portion of the sensor so as to make it difficult for the magnetization sensing portion 106 of the TMR sensor to detect. In this manner, the state shown in FIGS. 3A and 3B can be obtained. Any magnetic field application units for applying the direct-current magnetization may be used as long as a desired magnetic filed can be applied. A permanent magnet or an electromagnet may be used.

Generally, when the magnetic permeability under vacuum of a point, which is a distance r away from the center of a composite particle having magnetization m, is assumed to be is μ₀, the stray magnetic field Hₛ generating from the composite particles is expressed by the expression (1) below.

$$Expression 1$$

$$Hs = - \frac{1}{4 πμ {or}^{3}} [ m - \frac{3}{{r}^{2}} ( mr ) r ] \qquad ( 1 )$$

From Expression (1), the magnitude of the in-plane component of the stray magnetic field formed by composite particle A101 of 175 nm in diameter in the magnetization sensing portion 106 of the TMR sensor can be calculated. In consideration of the presence of a protecting film and wiring on the magnetization sensing portion 106 of the TMR sensor, the upper surface of the magnetization sensing portion 106 of the TMR sensor is assumed to be present at a 15 nm depth from the TMR sensor surface 100. Furthermore, when a target substance is detected based on the detection of Composite particle B102, composite particle A101 and the TMR sensor surface 100 come to sandwich a trapping substance and an antigen 104. The height thereof varies depending upon the type of antigen and antibody. The thickness of each of antibodies can be generally presumed to be 15 nm and the diameter of antigens is around several tens of nm. Depending upon the type of virus and bacteria or the state of an antibody, the height may be outside this range. However, assuming that the total height is about 50 nm, magnetic field distribution is as shown in FIG. 4.

Even in the state of FIG. 4, it can be confirmed that a detectable magnetic field is formed in at least near the projected plane of Composite particle B102 in the magnetization sensing portion 106 of the TMR sensor.

As is apparent from Expression (1), the magnetic field formed around the composite particle is in proportional to the magnetization of the composite particle and is attenuated in proportional to the third power of the distance from the composite particle. Therefore, composite particle A101 of the present invention, which has a large content of a magnetic substance and a thin shell surrounding the core, is suitable for detection by a magnetic sensor.

When a target substance to be detected by a biosensor is a non-magnetic substance, if Composite particle B102 of the present invention is fixed, the target substance can be indirectly detected by detection of the composite particle.

### Examples

The present invention will be more specifically described below by way of Examples; however, the present invention is not limited to these examples.

### (Synthesis of Magnetic Substance)

Compounds FeCl₃6H₂O and FeCl₂4H₂O were dissolved in water in an equimolar amount to obtain a solution. While the solution was placed at room temperature with stirring vigorously, 28% ammonia water was added to obtain a magnetite suspension solution. To the suspension solution, oleic acid was added and stirred at 70° C. for one hour and at 110° C. for one hour to obtain slurry. The slurry was washed with a large amount of water and ethanol and dried under reduced pressure to obtain powdery hydrophobic magnetite.

The hydrophobic magnetite thus obtained was evaluated under transmission electron microscope (TEM), a weight-average dry particle size (Dw) was 8 nm.

When hydrophobic magnetite was prepared in the same manner as above by using FeCl₃6H₂O and FeCl₂4H₂O in a molar ratio of 1:1.5, 1:2.5 and 1:5.0, hydrophobic magnetite having a Dw of 12 nm, 17 nm and 26 nm, respectively was obtained.

### (Synthesis of Polymer Compound 1)

Styrene and methacrylic acid were dissolved in toluene to obtain a solution and nitrogen bubbling was performed for 30 minutes. Thereafter, azobisisobutyronitrile was added to the solution and stirred at 60° C. for 2 hours. Subsequently, the solution was added dropwise to a large amount of methanol and precipitates were collected by filtration. In this manner, Polymer compound 1 having a hydrophobic portion derived from styrene and a hydrophilic portion derived from methacrylic acid was synthesized. Note that the hydrophilic portion derived from methacrylic acid is a carboxyl group.

Polymer compound 1 was evaluated by size exclusion chromatography (GPC). As a result, the weight-average molecular weight was 8200.

Furthermore, the solubility of the polymer compound to water was checked, it was insoluble in water in the pH range lower than pH 10 and soluble in water in the pH range of pH 10 or more.

### (Synthesis of Polymer Compound 2)

Styrene and 4-vinylpyridine were dissolved in toluene to obtain a solution and nitrogen bubbling was performed for 30 minutes. Thereafter, azobisisobutyronitrile was added to the solution and stirred at 60° C. for 2 hours. Subsequently, the solution was added dropwise to a large amount of methanol and precipitates were collected by filtration. In this manner, Polymer compound 2 having a hydrophobic portion derived from styrene and a hydrophilic portion derived from 4-vinylpyridine was synthesized. Note that the hydrophilic portion derived from 4-vinylpyridine is an amino group.

Polymer compound 2 was evaluated by size exclusion chromatography (GPC). As a result, the weight-average molecular weight was 6800.

Furthermore, the solubility of the polymer compound to water was checked, it was soluble in water in the pH range of pH 3 or less and insoluble in water in the pH range of more than pH 3.

### (Synthesis of Polymer Compound 3)

4-Chloromethylstyrene and methacrylic acid were dissolved in toluene to obtain a solution and nitrogen bubbling was performed for 30 minutes. Thereafter, azobisisobutyronitrile was added to the solution and stirred at 60° C. for 2 hours. Subsequently, the solution was added dropwise to a large amount of methanol and precipitates were collected by filtration. In this manner, Polymer compound 3 having a hydrophobic portion derived from 4-chloromethylstyrene and a hydrophilic portion derived from methacrylic acid was synthesized. Note that the hydrophilic portion derived from methacrylic acid is a carboxyl group.

Polymer compound 3 was evaluated by size exclusion chromatography (GPC). As a result, the weight-average molecular weight was 7300.

Furthermore, the solubility of the polymer compound to water was checked, it was insoluble in water in the pH range lower than pH 10 and soluble in water in the pH range of pH 10 or more.

### Example 1

Hydrophobic magnetite (3.0 g) having a Dw of 8 nm was dispersed in hexane (6 g) to prepare a hexane mixed solution. Subsequently, sodium dodecyl sulfate (SDS) (0.01 g) was dissolved in distilled water (30 g) to prepare an SDS aqueous solution. Furthermore, Polymer compound 1 (1 g) was dissolved in distilled water (50 g) controlled to pH 11 with sodium hydroxide to prepare an aqueous polymer compound solution.

The hexane mixed solution and the SDS aqueous solution were mixed to obtain a solution mixture. While the solution mixture was cooled by a cooling agent, the mixture was sheared by an ultrasonic homogenizer for 4 minutes to prepare an emulsion. The obtained emulsion was evaluated by DLS 8000. As a result, Dhw was 230 nm, Dhn was 211 nm and Dhw/Dhn was 1.09. It was confirmed that the emulsion is classified into a mini-emulsion.

To the obtained mini-emulsion, the aqueous polymer compound solution was added and stirred at room temperature for 30 minutes. Thereafter, ethanol (25 ml) was added for 30 minutes. Further, the temperature was increased to 70° C. and stirring was performed for one hour. Subsequently, the mini-emulsion was cooled to room temperature and a 0.03N HCl aqueous solution was added for 30 minutes until the pH of the mini-emulsion reached to 7.5. Further, the temperature was increased to 70° C. and stirring was performed for one hour to obtain a dispersion solution of Composite particle 1. Finally, the dispersion solution of Composite particle 1 was purified by dialysis against distilled water for 3 days.

The obtained Composite particle 1 was evaluated by TEM. As a result, Dw was 170 nm, Dn was 167 nm and Dw/Dn was 1.02. Since a coating layer of Polymer compound 1 on the surface of Composite particle 1 was not clearly confirmed in the image of TEM, it is presumed that Polymer compound 1 is not present on the surface of Composite particle 1 or forms an extremely thin film. Furthermore, evaluation was performed by TG-DTA (Thermogravimetry/Differential Thermal Analysis) (Thermo Plus manufactured by Rigaku Corp.). As a result, the magnetite content of Composite particle 1 was 82 wt %. Note that a TEM image of Composite particle 1 is shown in FIG. 5.

The dispersion solution of Composite particle 1 was evaluated by DLS 8000. As a result, a single-peak particle size distribution was obtained. Dhw was 172 nm, Dhn was 159 nm and Dhw/Dhn was 1.08. Furthermore, the electrophoretic mobility of the magnetic particle in an aqueous solution of pH 2 to 13 was evaluated by ZEECOM (Microtec Co., Ltd.). As a result, typical behavior to a particle having a carboxyl group was observed. From this, it was confirmed that a thin film of Polymer compound 1 is formed on the surface of Composite particle 1. Furthermore, Composite particle 1 was lyophilized and surface element analysis was performed by XPS. As a result, it was confirmed that Polymer compound 1 is present on the surface of Composite particle 1.

### Example 2

Hydrophobic magnetite (3.0 g) having a Dw of 8 nm was dispersed in hexane (6 g) to prepare a hexane mixed solution. Subsequently, SDS (0.01 g) was dissolved in distilled water (30 g) to prepare an SDS aqueous solution. Furthermore, Polymer compound 2 (1 g) was dissolved in distilled water (50 g) controlled to pH 1.5 with an aqueous hydrochloric acid solution to prepare an aqueous polymer compound solution.

The hexane mixed solution and the SDS aqueous solution were mixed to obtain a solution mixture. While the solution mixture was cooled by a cooling agent, the mixture was sheared by an ultrasonic homogenizer for 4 minutes to prepare an emulsion. The obtained mini-emulsion was evaluated by DLS 8000. As a result, Dhw was 217 nm, Dhn was 197 nm and Dhw/Dhn was 1.10. It was confirmed that the emulsion is classified into a mini-emulsion.

To the obtained mini-emulsion, the aqueous polymer compound solution was added, and stirred at room temperature for 30 minutes. Thereafter, ethanol (25 ml) was added for 30 minutes. Further, the temperature was increased to 70° C. and stirring was performed for one hour. Subsequently, the mini-emulsion was cooled to room temperature and a 0.03N NaOH aqueous solution was added for 30 minutes until the pH of the mini-emulsion reached to 6.0. Further, the temperature was increased to 70° C. and stirring was performed for one hour to obtain a dispersion solution of Composite particle 2. Finally, the dispersion solution of Composite particle 2 was purified by dialysis against distilled water for 3 days.

The obtained Composite particle 2 was evaluated by TEM. As a result, Dw was 166 nm, Dn was 150 nm and Dw/Dn of 1.11. Since a coating layer of Polymer compound 2 on the surface of Composite particle 2 was not clearly confirmed in the image of TEM, it is presumed that Polymer compound 2 is not present on the surface of Composite particle 2 or forms an extremely thin film. Furthermore, evaluation was performed by TG-DTA. As a result, the magnetite content of Composite particle 2 was 81 wt %.

The dispersion solution of Composite particle 2 was evaluated by DLS 8000. As a result, a single-peak particle size distribution was obtained. Dhw was 165 nm, Dhn was 147 nm and Dhw/Dhn was 1.12. Furthermore, the electrophoretic mobility of Composite particle 2 in an aqueous solution of pH 2 to 13 was evaluated by ZEECOM. As a result, typical behavior to a particle having an amino group was observed. From this, it was confirmed that a thin film of Polymer compound 2 is formed on the surface of Composite particle 2. Furthermore, Composite particle 2 was lyophilized and surface element analysis was performed by XPS. As a result, it was confirmed that Polymer compound 2 is present on the surface of Composite particle 2.

### Example 3

Experiment was performed under the same conditions as in Example 1 except that hydrophobic magnetite having a Dw of 12 nm was used. Also in this case, it was confirmed that an emulsion that can be classified into a mini-emulsion can be obtained.

The obtained Composite particle 3 was evaluated by TEM. As a result, Dw was 181 nm, Dn was 163 nm and Dw/Dn was 1.11. Since a coating layer of Polymer compound 1 on the surface of Composite particle 3 was not clearly confirmed in the image of TEM, it is presumed that Polymer compound 1 is not present on the surface of Composite particle 3 or forms an extremely thin film. Furthermore, evaluation was performed by TG-DTA (Thermogravimetry/Differential Thermal Analysis). As a result, the magnetite content of Composite particle 3 was 85 wt %.

The dispersion solution of Composite particle 3 was evaluated by DLS 8000. As a result, a single-peak particle size distribution was obtained. Dhw was 184 nm, Dhn was 166 nm and Dhw/Dhn was 1.11. Furthermore, the electrophoretic mobility of Composite particle 3 in an aqueous solution of pH 2 to 13 was evaluated by ZEECOM. As a result, typical behavior to a particle having a carboxyl group was observed. From this, it was confirmed that a thin film of Polymer compound 1 is formed on the surface of Composite particle 3. Furthermore, Composite particle 3 was lyophilized and surface element analysis was performed by XPS. As a result, it was confirmed that Polymer compound 1 is present on the surface of Composite particle 3.

### Example 4

Experiment was performed under the same conditions as in Example 1 except that hydrophobic magnetite having a Dw of 17 nm was used. Also in this case, it was confirmed that an emulsion classified into a mini-emulsion can be obtained.

The obtained Composite particle 4 was evaluated by TEM. As a result, Dw was 194 nm, Dn was 175 nm and Dw/Dn was 1.11. Since a coating layer of Polymer compound 1 on the surface of Composite particle 4 was not clearly confirmed in the image of TEM, it is presumed that Polymer compound 1 is not present on the surface of Composite particle 4 or forms an extremely thin film. Furthermore, evaluation was performed by TG-DTA (Thermogravimetry/Differential Thermal Analysis). As a result, the magnetite content of Composite particle 4 was 86 wt %.

The dispersion solution of Composite particle 4 was evaluated by DLS 8000. As a result, a single-peak particle size distribution was obtained. Dhw was 192 nm, Dhn was 170 nm and Dhw/Dhn was 1.13. Furthermore, the electrophoretic mobility of Composite particle 4 in an aqueous solution of pH 2 to 13 was evaluated by ZEECOM. As a result, typical behavior to a particle having a carboxyl group was observed. From this, it was confirmed that a thin film of Polymer compound 1 is formed on the surface of Composite particle 4. Furthermore, Composite particle 4 was lyophilized and surface element analysis was performed by XPS. As a result, it was confirmed that Polymer compound 1 is present on the surface of Composite particle 4.

### Example 5

Experiment was performed under the same conditions as in Example 1 except that hydrophobic magnetite (2.0 g) and SDS (0.02 g) were used. Also in this case, it was confirmed that an emulsion classified into a mini-emulsion can be obtained.

The obtained Composite particle 5 was evaluated by TEM. As a result, Dw was 52 nm, Dn was 46 nm and Dw/Dn was 1.12. Since a coating layer of Polymer compound 1 on the surface of Composite particle 5 was not clearly confirmed in the image of TEM, it is presumed that Polymer compound 1 is not present on the surface of Composite particle 5 or forms an extremely thin film. Furthermore, evaluation was performed by TG-DTA (Thermogravimetry/Differential Thermal Analysis). As a result, the magnetite content of Composite particle 5 was 80 wt %.

The dispersion solution of Composite particle 5 was evaluated by DLS 8000. As a result, a single-peak particle size distribution was obtained. Dhw was 52 nm, Dhn was 46 nm and Dhw/Dhn was 1.14. Furthermore, the electrophoretic mobility of Composite particle 5 in an aqueous solution of pH 2 to 13 was evaluated by ZEECOM. As a result, typical behavior to a particle having a carboxyl group was observed. From this, it was confirmed that a thin film of Polymer compound 1 is formed on the surface of Composite particle 5. Furthermore, Composite particle 5 was lyophilized and surface element analysis was performed by XPS. As a result, it was confirmed that Polymer compound 1 is present on the surface of Composite particle 5.

### Example 6

Experiment was performed under the same conditions as in Example 1 except that hydrophobic magnetite (4.5 g) was used. Also in this case, it was confirmed that an emulsion classified into a mini-emulsion can be obtained.

The obtained Composite particle 6 was evaluated by TEM. As a result, Dw was 282 nm, Dn was 245 nm and Dw/Dn was 1.15. Since a coating layer of Polymer compound 1 on the surface of Composite particle 6 was not clearly confirmed in the image of TEM, it is presumed that Polymer compound 1 is not present on the surface of Composite particle 6 or forms an extremely thin film. Furthermore, evaluation was performed by TG-DTA (Thermogravimetry/Differential Thermal Analysis). As a result, the magnetite content of Composite particle 6 was 82 wt %.

The dispersion solution of Composite particle 6 was evaluated by DLS 8000. As a result, a single-peak particle size distribution was obtained. Dhw was 286 nm, Dhn was 247 nm and Dhw/Dhn was 1.16. Furthermore, the electrophoretic mobility of Composite particle 6 in an aqueous solution of pH 2 to 13 was evaluated by ZEECOM. As a result, typical behavior to a particle having a carboxyl group was observed. From this, it was confirmed that a thin film of Polymer compound 1 is formed on the surface of Composite particle 6. Furthermore, Composite particle 6 was lyophilized and surface element analysis was performed by XPS. As a result, it was confirmed that Polymer compound 1 is present on the surface of Composite particle 6.

### Example 7

Hydrophobic magnetite (3.0 g) having a Dw of 8 nm was dispersed in styrene (1.5 g) and chloroform (4.5 g) to prepare a styrene/chloroform mixed solution. Subsequently, sodium dodecyl sulfate SDS (0.01 g) was dissolved in distilled water (30 g) to prepare an SDS aqueous solution. Furthermore, Polymer compound 1 (1 g) was dissolved in distilled water (50 g) controlled to pH 11 with sodium hydroxide to prepare an aqueous polymer compound solution.

The styrene/chloroform mixed solution and the SDS aqueous solution were mixed to obtain a solution mixture. While the solution mixture was cooled by a cooling agent, the mixture was sheared by an ultrasonic homogenizer for 4 minutes to prepare an emulsion. The obtained mini-emulsion was evaluated by DLS 8000. As a result, Dhw was 172 nm, Dhn was 158 nm and Dhw/Dhn was 1.09. It was confirmed that the emulsion is classified into a mini-emulsion.

To the obtained mini-emulsion, the aqueous polymer compound solution was added, and stirred at room temperature for 30 minutes. Thereafter, chloroform was selectively extracted from the mini-emulsion by use of an evaporator. After completion of chloroform extraction, the mini-emulsion was bubbled with nitrogen for 30 minutes. The temperature was increased to 70° C. and potassium persulfate was added and stirred for 6 hours to obtain Composite particle 7. Finally, the dispersion solution of Composite particle 7 was dialyzed against distilled water for 3 days and the pH was adjusted to 6.5.

The obtained Composite particle 7 was evaluated by TEM. As a result, Dw was 151 nm, Dn was 134 nm and Dw/Dn of 1.12. Since a coating layer of Polymer compound 1 on the surface of Composite particle 7 was not clearly confirmed in the image of TEM, it is presumed that Polymer compound 1 is not present on the surface of magnetic particle or forms an extremely thin film. Furthermore, evaluation was performed by TG-DTA (Thermogravimetry/Differential Thermal Analysis). As a result, the magnetite content of Composite particle 7 was 53 wt %.

The dispersion solution of Composite particle 7 was evaluated by DLS 8000. As a result, a single-peak particle size distribution was obtained. Dhw was 154 nm, Dhn was 132 nm and Dhw/Dhn was 1.17. Furthermore, the electrophoretic mobility of Composite particle 7 in an aqueous solution of pH 2 to 13 was evaluated by ZEECOM. As a result, typical behavior to a particle having a carboxyl group was observed. From this, it was confirmed that a thin film of Polymer compound 1 is formed on the surface of Composite particle 7. Furthermore, Composite particle 7 was lyophilized and surface element analysis was performed by XPS. As a result, it was confirmed that Polymer compound 1 is present on the surface of Composite particle 7.

### Example 8

Experiment was performed under the same conditions as in Example 1 except that hydrophobic platinum-iron particles (manufactured by Toda Kogyo Corp.) were used as a magnetic substance.

The hydrophobic platinum-iron particles are formed by modifying the surface of platinum-iron particles with a surfactant (not disclosed) to impart hydrophobicity and exhibit good dispersibility to a hydrophobic organic solvent such as hexane. The particles were evaluated by a transmission electron microscope (TEM). As a result, weight-average dry particle size (Dw) was 4 nm.

Also in this case, it was confirmed that an emulsion classified into a mini-emulsion can be obtained.

The obtained Composite particle 8 was evaluated by TEM. As a result, Dw was 191 nm, Dn was 175 nm and Dw/Dn was 1.09. Since a coating layer of Polymer compound 1 on the surface of Composite particle 8 was not clearly confirmed in the image of TEM, it is presumed that Polymer compound 1 is not present on the surface of Composite particle 8 or forms an extremely thin film. Furthermore, evaluation was performed by TG-DTA (Thermogravimetry/Differential Thermal Analysis). As a result, the magnetite content of Composite particle 8 was 76 wt %.

The dispersion solution of Composite particle 8 was evaluated by DLS 8000. As a result, a single-peak particle size distribution was obtained. Dhw was 194 nm, Dhn was 178 nm and Dhw/Dhn was 1.09. Furthermore, the electrophoretic mobility of Composite particle 8 in an aqueous solution of pH 2 to 13 was evaluated by ZEECOM. As a result, typical behavior to a particle having a carboxyl group was observed. From this, it was confirmed that a thin film of Polymer compound 1 is formed on the surface of Composite particle 8. Furthermore, Composite particle 8 was lyophilized and surface element analysis was performed by XPS. As a result, it was confirmed that Polymer compound 1 is present on the surface of Composite particle 8.

### Example 9

Hydrophobic magnetite (3.0 g) having a Dw of 8 nm was dispersed in a mixed organic solvent containing hexane (3 g) and chloroform (3 g) to prepare a hexane/chloroform mixed solution. Subsequently, sodium dodecyl sulfate SDS (0.01 g) was dissolved in distilled water (30 g) to prepare an SDS aqueous solution. Furthermore, Polymer compound 1 (1 g) was dissolved in distilled water (50 g) controlled to pH 11 with sodium hydroxide to prepare an aqueous polymer compound solution.

The hexane/chloroform mixed solution and the SDS aqueous solution were mixed to obtain a solution mixture. While the solution mixture was cooled by a cooling agent, the mixture was sheared by an ultrasonic homogenizer for 4 minutes to prepare an emulsion. The obtained mini-emulsion was evaluated by DLS 8000. As a result, Dhw was 205 nm, Dhn was 184 nm and Dhw/Dhn was 1.11. It was confirmed that the emulsion is classified into a mini-emulsion.

To the obtained mini-emulsion, the aqueous polymer compound solution was added, and stirred at room temperature for 30 minutes. Thereafter, ethanol (25 ml) was added for 30 minutes. Further, the temperature was increased to 70° C. and stirring was performed for one hour. Subsequently, the mini-emulsion was cooled to room temperature and a 0.03N HCl aqueous solution was added for 30 minutes until the pH of the mini-emulsion reached to 7.5. Further, the temperature was increased to 70° C. and stirring was performed for one hour to obtain a dispersion solution of Composite particle 9. Finally, the dispersion solution of Composite particle 9 was purified by dialysis against distilled water for 3 days.

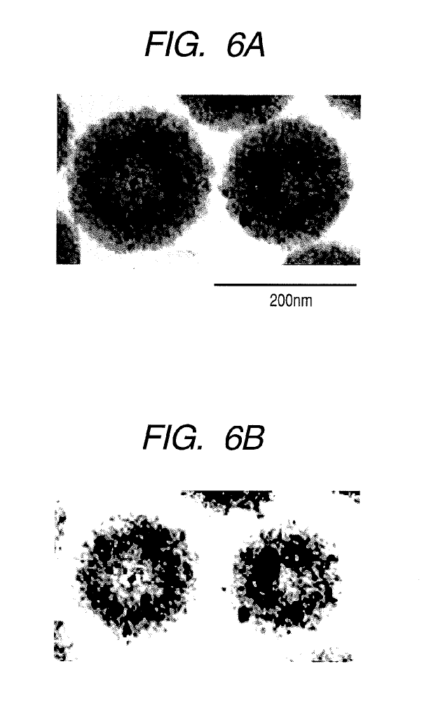

The obtained Composite particle 9 was evaluated by TEM. As a result, Dw was 181 nm, Dn was 160 nm and Dw/Dn of 1.13, and it was confirmed that a hollow structure is formed inside. Since a coating layer of Polymer compound 1 on the surface of Composite particle 9 was not clearly confirmed in the image of TEM, it is presumed that Polymer compound 1 is not present on the surface of Composite particle 9 or forms an extremely thin film. Furthermore, evaluation was performed by TG-DTA (Thermogravimetry/Differential Thermal Analysis). As a result, the magnetite content of Composite particle 9 was 81 wt %. Note that a TEM image of Composite particle 9 is shown in FIGS. 6A and 6B. FIG. 6A is a transmission electron micrograph of composite particles obtained in Example 9. FIG. 6B is a transmission electron micrograph of FIG. 6A enhanced contrast by processing the image.

The Example differs from Example 1 in that as an organic solvent for dispersing magnetite (3.0 g), a solvent mixture containing chloroform (3 g) and hexane (3 g) is used. Since chloroform has large hydrophilicity compared to hexane, a fractionation rate is larger in this example using a solvent mixture, compared to in Example 1 using hexane alone as the organic solvent. Because of this, a hollow structure shown in FIGS. 6A and 6B is presumably generated.

The dispersion solution of Composite particle 9 was evaluated by DLS 8000. As a result, a single-peak particle size distribution was obtained. Dhw was 186 nm, Dhn was 160 nm and Dhw/Dhn was 1.16. Furthermore, the electrophoretic mobility of Composite particle 9 in an aqueous solution of pH 2 to 13 was evaluated by ZEECOM (Microtec Co., Ltd.). As a result, typical behavior to a particle having a carboxyl group was observed. From this, it was confirmed that a thin film of Polymer compound 1 is formed on the surface of Composite particle 9. Furthermore, Composite particle 9 was lyophilized and surface element analysis was performed by XPS. As a result, it was confirmed that Polymer compound 1 is present on the surface of Composite particle 9.

### Example 10

Experiment was performed under the same conditions as in Example 1 except that Polymer compound 3 was used in place of Polymer compound 1. Also in this case, it was confirmed that an emulsion classified into a mini-emulsion can be obtained.

The obtained Composite particle 10 was evaluated by TEM. As a result, Dw was 208 nm, Dn was 194 nm and Dw/Dn was 1.07. Since a coating layer of Polymer compound 3 on the surface of Composite particle 10 was not clearly confirmed in the image of TEM, it is presumed that Polymer compound 3 is not present on the surface of Composite particle 10 or forms an extremely thin film. Furthermore, evaluation was performed by TG-DTA (Thermogravimetry/Differential Thermal Analysis). As a result, the magnetite content of Composite particle 10 was 84 wt %.

The dispersion solution of Composite particle 10 was evaluated by DLS 8000. As a result, a single-peak particle size distribution was obtained. Dhw was 221 nm, Dhn was 204 nm and Dhw/Dhn was 1.08. Furthermore, the electrophoretic mobility of Composite particle 10 in an aqueous solution of pH 2 to 13 was evaluated by ZEECOM (Microtec Co., Ltd.). As a result, typical behavior to a particle having a carboxyl group was observed. From this, it was confirmed that a thin film of Polymer compound 3 is formed on the surface of Composite particle 10. Furthermore, Composite particle 10 was lyophilized and surface element analysis was performed by XPS. As a result, a signal derived from a chloro group was observed. Form this, it was confirmed that Polymer compound 3 is present on the surface of Composite particle 10.

Next, Composite particle 10 (1.0 g) was dispersed in distilled water (20 g). Thereafter, an aqueous solution of sodium N,N-dimethyldithiocarbamate (0.5 g) dissolved in distilled water (5 g) was added to react a chloro group exposed on the surface of the particle with sodium N,N-dimethyldithiocarbamate in water. As a result, Composite particle 11 was obtained having an N,N-dimethyldithiocarbamate group introduced in the surface. When Composite particle 11 was subjected to FT-IR measurement (manufactured by Perkin Elmer), the C═S stretching vibration peak derived from the N,N-dimethyldithiocarbamate group was observed. Furthermore, Composite particle 11 was lyophilized and surface element analysis was performed by XPS. As a result, a signal derived from atom S was observed on the surface. From these analyses, it was confirmed that an N,N-dimethyldithiocarbamate group was adsorbed to the surface of composite particles.

The obtained Composite particle 11 (0.25 g) was dispersed in distilled water (100 g) under light-tight conditions. Thereafter, 0.1 mmol N,N-diethyldithiocarbamide acetic acid as a free polymerization initiator, 100 mmol N-methacryloyloxyethyl-N,N-dimethyl ammonium-α-N-methylcarboxybetain (hereinafter MCB) as a monomer were added and nitrogen bubbling was performed for 30 minutes or more to remove oxygen within the reaction system. Thereafter, the dispersion solution was irradiated with UV rays by a high-pressure mercury lamp (400 W, manufactured by Riko Kagakusangyou Kabushiki Kaisha). After UV irradiation was performed for 40 minutes, centrifugal separation was repeatedly performed to remove excessive MCB and a free polymer, and washing was performed with distilled water to obtain a water dispersion solution of Composite particle 12 having poly(MCB) grafted thereto.

The dispersion solution of Composite particle 12 was evaluated by DLS 8000. As a result, it was confirmed that a particle size increases by poly(MCB) grafting compared to Composite particle 11. Dhw was 268 nm, Dhn was 244 nm and Dhw/Dhn was 1.10.

The molecular weight of a polymer produced from N,N-diethyldithiocarbamide acetic acid, which was added as a free polymerization initiator, and a molecular weight distribution thereof were measured. As a result, the number-average molecular weight was 1.17×10⁵ and the molecular weight distribution was 1.38. From this, it is presumed that poly(MCB) grafted on the surface of Composite particle 12 is a polymer compound having a uniform chain length.

In a 0.01M PBS buffer solution, 0.1 wt % dispersion solution of Composite particle 12 was evaluated by DLS-8000 and FPAR-1000 (manufactured by Otsuka Electronics Co., Ltd.). As a result, a single-peak particle size distribution was obtained. From this, it was confirmed that high dispersion stability is obtained.

Furthermore, in a 0.01M PBS buffer solution, the adsorption amount of bovine serum albumin (hereinafter BSA) to Composite particle 12 was evaluated by UV/visible light spectrophotometer (manufactured by Perkin Elmer). As a result, it was confirmed that the BSA adsorption amount to Composite particle 12 is significantly suppressed compared to Composite particle 11.

### Example 11

Experiment was performed under the same conditions as in Example 1 except that Polymer compound 3 was used in place of Polymer compound 1. Also in this case, it was confirmed that an emulsion classified into a mini-emulsion can be obtained.

The obtained Composite particle 13 was evaluated by TEM. As a result, Dw was 208 nm, Dn was 194 nm and Dw/Dn was 1.07. Since a coating layer of Polymer compound 3 on the surface of Composite particle 13 was not clearly confirmed in the image of TEM, it is presumed that Polymer compound 3 is not present on the surface of composite particle or forms an extremely thin film. Furthermore, evaluation was performed by TG-DTA (Thermogravimetry/Differential Thermal Analysis). As a result, the magnetite content of Composite particle 13 was 84 wt %.

The dispersion solution of Composite particle 13 was evaluated by DLS 8000. As a result, a single-peak particle size distribution was obtained. Dhw was 221 nm, Dhn was 204 nm and Dhw/Dhn was 1.08. Furthermore, the electrophoretic mobility of Composite particle 13 in an aqueous solution of pH 2 to 13 was evaluated by ZEECOM (Microtec Co., Ltd.). As a result, typical behavior to a particle having a carboxyl group was observed. From this, it was confirmed that a thin film of Polymer compound 3 is formed on the surface of Composite particle 13. Furthermore, Composite particle 13 was lyophilized and surface element analysis was performed by XPS. As a result, a signal derived from a chloro group was observed. Form this, it was confirmed that polymer compound 3 is present on the surface of Composite particle 13.

Next, the water dispersion solution containing

Composite particle 13 (0.25 g) was dialyzed to replace the dispersion medium by methanol. After Composite particle 13 was dispersed again, 0.10 mM benzyl chloride was added as a free polymerization initiator, and thereafter 0.10 mM CuCl and 0.30 mM 2,2′-bipyridyl were added. Nitrogen bubbling was performed for 30 minutes or more to remove oxygen within the reaction system and the reaction system was purged with nitrogen. Then, 100 mM MCB was added as a monomer and atom-transfer radical polymerization was performed at 40° C. for 4 hours. After the reaction, centrifugal separation was repeatedly performed to remove a copper complex added as a catalyst and excessive MCB, and washing was performed with methanol followed by distilled water to obtain a water dispersion solution of Composite particle 14 having poly(MCB) grafted thereto.

The dispersion solution of Composite particle 14 was evaluated by DLS 8000. Dhw was 262 nm, Dhn was 241 nm and Dhw/Dhn was 1.09.

Furthermore, the molecular weight of a polymer produced from benzyl chloride, which was added as a free polymerization initiator, and a molecular weight distribution thereof were measured. As a result, the number-average molecular weight was 1.02×10⁵ and the molecular weight distribution was 1.27. From this, it is presumed that poly(MCB) grafted on the surface of Composite particle 14 is a polymer compound having a uniform chain length.

In a 0.01M PBS buffer solution, 0.1 wt % Composite particle 14 dispersion solution was evaluated by FPAR-1000. As a result, a single-peak particle size distribution was obtained. From this, it was confirmed that high dispersion stability is obtained.

Furthermore, in a 0.01M PBS buffer solution, the absorption amount of BSA to Composite particle 14 was evaluated by UV/visible light spectrophotometer (manufactured by Perkin Elmer). As a result, it was confirmed that the BSA adsorption amount to Composite particle 14 is significantly suppressed compared to Composite particle 13.

### Example 12

Composite particle 1 was dispersed in a solution mixture containing an aqueous solution of N-hydroxysulfosuccinimide and an aqueous solution of 1-ethyl-3-(3-dimethylaminopropyl)carbodiimide hydrochloride. To the dispersion solution, a solution having anti-lysozyme (Rabbit-Poly) dissolved in a phosphate buffer was further added. In this manner, anti-lysozyme (Rabbit-Poly) was chemically adsorbed to Composite particle 1 to obtain Composite particle 15.

The dispersion solution of Composite particle 15 was evaluated by DLS 8000. As a result, a single-peak particle size distribution was obtained. Dhw was 176 nm, Dhn was 162 nm and Dhw/Dhn was 1.09.

### Example 13

The magnetic particle obtained by grafting poly (MCB) to Composite particle 12 was dispersed in distilled water. To the distilled water, further a solution having succinimidyl biotin dissolved in N,N′-dimethylformamide was further added. In this manner, succinimidyl biotin was chemically adsorbed to Composite particle 12 to obtain Composite particle 16.

The dispersion solution of Composite particle 16 was evaluated by DLS 8000. As a result, a single-peak particle size distribution was obtained. Dhw was 276 nm, Dhn was 240 nm and Dhw/Dhn was 1.15.

### Example 14

Composite particle 1 was dispersed in a solution mixture containing an aqueous solution of N-hydroxysulfosuccinimide and an aqueous solution of 1-ethyl-3-(3-dimethylaminopropyl)carbodiimide hydrochloride. To the dispersion solution, a solution having streptavidin dissolved in a phosphate buffer was further added. In this manner, streptavidin was chemically adsorbed to Composite particle 1 to obtain Composite particle 17.

The dispersion solution of Composite particle 17 was evaluated by DLS 8000. As a result, a single-peak particle size distribution was obtained. Dhw was 192 nm, Dhn was 169 nm and Dhw/Dhn was 1.14.

### Example 15

Composite particle 1 was dispersed in a solution mixture containing an aqueous solution of N-hydroxysulfosuccinimide and an aqueous solution of 1-ethyl-3-(3-dimethylaminopropyl)carbodiimide hydrochloride. To the dispersion solution, a solution having 5′-end aminated DNA (15 mer) dissolved in a phosphate buffer was further added. In this manner, 5′-end aminated DNA (15 mer) was chemically adsorbed to Composite particle 1 to obtain Composite particle 18.

The dispersion solution of Composite particle 18 was evaluated by DLS 8000. As a result, a single-peak particle size distribution was obtained. Dhw was 174 nm, Dhn was 151 nm and Dhw/Dhn was 1.15.

### Example 16

Composite particle 1 was dispersed in a solution mixture containing an aqueous solution of N-hydroxysulfosuccinimide and an aqueous solution of 1-ethyl-3-(3-dimethylaminopropyl)carbodiimide hydrochloride. To the dispersion solution, a solution having anti-lysozyme (Mouse-Mono) dissolved in a phosphate buffer was further added. In this manner, anti-lysozyme (Mouse-Mono) was chemically adsorbed to Composite particle 1 to obtain Composite particle 19.

The dispersion solution of Composite particle 19 was evaluated by DLS 8000. As a result, a single-peak particle size distribution was obtained. Dhw was 181 nm, Dhn was 157 nm and Dhw/Dhn was 1.19.

### Example 17

Composite particle 1 was dispersed in a solution mixture formed of an aqueous solution of N-hydroxysulfosuccinimide and an aqueous solution of 1-ethyl-3-(3-dimethylaminopropyl)carbodiimide hydrochloride. To the dispersion solution, a solution having hen's egg lysozyme (HEL) dissolved in a phosphate buffer was further added. In this manner, hen's egg lysozyme (HEL) was chemically adsorbed to Composite particle 1 to obtain Composite particle 20.

The dispersion solution of Composite particle 20 was evaluated by DLS 8000. As a result, a single-peak particle size distribution was obtained. Dhw was 178 nm, Dhn was 153 nm and Dhw/Dhn was 1.16.

### Example 18

Composite particle 1 was dispersed in a solution mixture formed of an aqueous solution of N-hydroxysulfosuccinimide and an aqueous solution of 1-ethyl-3-(3-dimethylaminopropyl)carbodiimide hydrochloride. To the dispersion solution, a solution having aminoethanethiol dissolved in a phosphate buffer was further added. In this manner, aminoethanethiol was chemically adsorbed to Composite particle 1 to obtain Composite particle A.

The dispersion solution of Composite particle A was evaluated by DLS 8000. As a result, a single-peak particle size distribution was obtained. Dhw was 181 nm, Dhn was 154 nm and Dhw/Dhn was 1.18.

### Example 19

Composite particle 1 was dispersed in a solution mixture formed of an aqueous solution of N-hydroxysulfosuccinimide and an aqueous solution of 1-ethyl-3-(3-dimethylaminopropyl)carbodiimide hydrochloride. To the dispersion solution, a solution having anti-PSA dissolved in a phosphate buffer was further added. In this manner, anti-PSA was chemically adsorbed to Composite particle 1 to obtain Composite particle B.

The dispersion solution of Composite particle B was evaluated by DLS 8000. As a result, a single-peak particle size distribution was obtained. Dhw was 189 nm, Dhn was 162 nm and Dhw/Dhn was 1.17.

### Example 20

Hydrophobic magnetite (0.24 g) having a Dw of 8 nm was dispersed in hexane (60 g) to prepare a hexane mixed solution. Subsequently, sodium dodecyl sulfate SDS (0.6 g) was dissolved in distilled water (1000 g) to prepare an SDS aqueous solution. Furthermore, polymer compound 1 (2 g) was dissolved in distilled water (50 g) controlled to pH 11 with sodium hydroxide to prepare an aqueous polymer compound solution.

The hexane mixed solution and the SDS aqueous solution were subjected to membrane emulsification performed by a high-speed minikit (manufactured by SPG techno) to prepare an emulsion. The obtained emulsion was evaluated by an optical microscope. As a result, Dhw was 1020 nm, Dhn was 887 nm and Dhw/Dhn was 1.15. It was confirmed that a mono-dispersion emulsion is obtained.

To the obtained emulsion, the aqueous polymer compound solution was added and stirred at room temperature for 30 minutes and then stirred at 40° C. for 7 days to obtain a dispersion solution of Composite particle 20. Finally, the dispersion solution of Composite particle 20 was dialyzed against distilled water for 3 days to perform purification.

The obtained Composite particle 20 was evaluated by TEM. As a result, Dw was 192 nm, Dn was 160 nm and Dw/Dn was 1.20. Since a coating layer of polymer compound 1 on the surface of Composite particle 20 was not clearly confirmed in the image of TEM, it is presumed that polymer compound 1 is not present on the surface of Composite particle 20 or forms an extremely thin film. Furthermore, evaluation was performed by TG-DTA (Thermogravimetry/Differential Thermal Analysis) (Thermo Plus manufactured by Rigaku Corp.). As a result, the magnetite content of Composite particle 20 was 81 wt %.

The dispersion solution of Composite particle 20 was evaluated by DLS 8000. As a result, a single-peak particle size distribution was obtained. Dhw was 208 nm, Dhn was 170 nm and Dhw/Dhn was 1.22. Furthermore, the electrophoretic mobility of the magnetic particle in an aqueous solution of pH 2 to 13 was evaluated by ZEECOM (Microtec Co., Ltd.). As a result, typical behavior to a particle having a carboxyl group was observed. From this, it was confirmed that a thin film of polymer compound 1 is formed on the surface of Composite particle 20. Furthermore, Composite particle 20 was lyophilized and surface element analysis was performed by XPS. As a result, it was confirmed that polymer compound 1 is present on the surface of Composite particle 20.

### Comparative Example 1

Experiment was performed under the same conditions as in Example 1 except that hydrophobic magnetite having a Dw of 26 nm was used. In this case, a good mono-dispersion emulsion was not formed and the obtained magnetic particles were aggregated.

### Comparative Example 2

Experiment was performed under the same conditions as in Example 1 except that hydrophobic magnetite (5.5 g) was used. In this case, a good mono-dispersion emulsion was not formed.

The obtained magnetic particle was evaluated by TEM. As a result, Dw was 347 nm, Dn was 275 nm and Dw/Dn was 1.26. A sufficient mono-dispersity was not attained. Furthermore, the dispersion solution of the magnetic particle was evaluated by DLS 8000. As a result, a single-peak particle size distribution was not obtained. From this, it was confirmed that dispersibility of the magnetic particle is not sufficient.

### Example 21

### (i) Magnetic Sensor

In this example, detection by Composite particle A101 using a TMR element will be described.

As the TMR element used in this example, a multi-layered film was used, which was formed by sequentially stacking, on a Si substrate, a multi-layered film formed of a Ta film, a Cu film and a Ta film as a underlying film, a multi-layered film formed of a PtMn film, a CoFe film, a Ru film and a CoFeB film, as a lower magnetic film, an MgO film as a spin tunnel film and a CoFeB film, as an upper magnetic film serving as a magnetic sensing portion.

On the upper magnetic film, a Pt film as a protecting film and upper wiring for supplying a detection current are arranged. Furthermore, an Au film is prepared on the sensor surface 100.

In this example, a TMR sensor having the aforementioned structure and magnetic anisotropy in the element in-plane direction is used. Therefore, the TMR sensor has a feature in that the magnetic filed in the element in-plane direction can be detected and the electric resistance of the element varies depending upon the magnitude of the magnetic field.

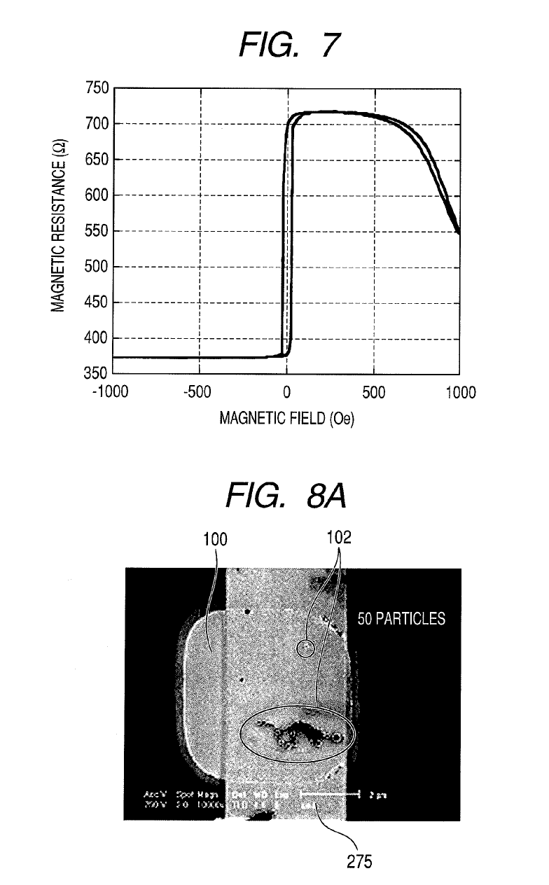

In this example, a TMR sensor having an upper-surface area of the element: 6 μm×6 μm and a change rate of magnetic resistance when a magnetic field is applied in the easy-axis direction: about 100% (FIG. 7) is used.

### (ii) Fixation of Composite Particle on Sensor

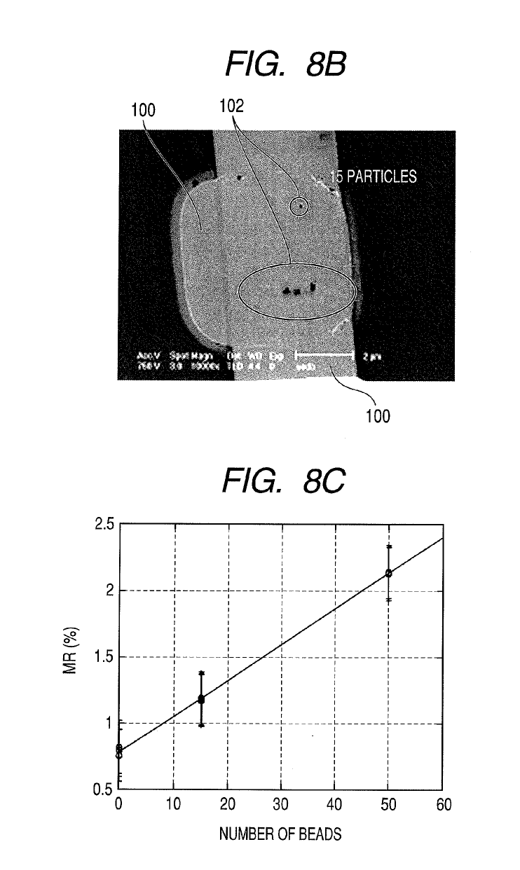

Composite particles A101 having a thiol surface according to Example 18 is diluted by EtOH solvent and supplied dropwise on the surface 100 of the TMR sensor. After immobilized with Au—SH, the surface 100 of the TM sensor is washed to fix composite particles on the TM sensor surface 100 (FIG. 8A).

(iii) Detection of Composite Particle

External magnetic field H_{⊥} of 1 kOe, is applied in the direction perpendicular to the sensor surface, which is the direction having difficulty with detection for the TMR sensor used herein to detect. By this operation, the direction of magnetization of Composite particles A102 is aligned virtually perpendicular to the film surface. In the conditions, when an electric resistance of the element was measured, the element resistance, which reflects the stray magnetic field from Composite particle B102, can be obtained. The measurement value is compared to element resistance measured in the absence of Composite particles B102. In this manner, the presence/absence or concentration of the composite particles can be detected.

FIG. 8C shows a change of TMR element resistance depending upon the number of fixed particles. This is obtained by measuring with respect to the cases where 50 Composite particles A (FIG. 8A) are fixed and 15 Composite particles A (FIG. 8B) are fixed on the sensor in comparison with the case having no fixed particles. FIG. 8A is a transmission electron micrograph showing fixation of Composite particle B and an output of the corresponding TMR sensor. FIG. 8B is a transmission electron micrograph showing fixation of Composite particle B and an output of the corresponding TMR sensor.

The vertical axis indicates the ratio of element resistance when a magnetic field H_{⊥} (1 kOe) is applied in the perpendicular direction to the sensor surface, that is, the change ratio (%) of a resistance value (R_{H⊥=1k}) at H_{⊥}=1 kOe relative to a resistance value (R_{H⊥=0}) at H_{⊥}=0, which is expressed by (R_{H⊥=1000}−R_{H⊥=0})/R_{H⊥=0}. It was confirmed that an increase of the output reflects the number of particles. In this manner, the number of fixed particles can be read out based on an output change of the TMR sensor.

### Example 22

Next, in this example, detection of a prostate specific antigen will be described, which is performed through detection of Composite particle B102 by the TMR element.

### i) Magnetic Sensor

On the surface 100 of the same TMR sensor as used in Example 21, a primary antibody 103(b) corresponding to a target substance 104 is prepared. In the region except the surface 100 of the TMR sensor that may be in contact with a test substance, a non-specific adhesion preventing material corresponding to the target substance is prepared.

### ii) Fixation of Composite Particle on Sensor

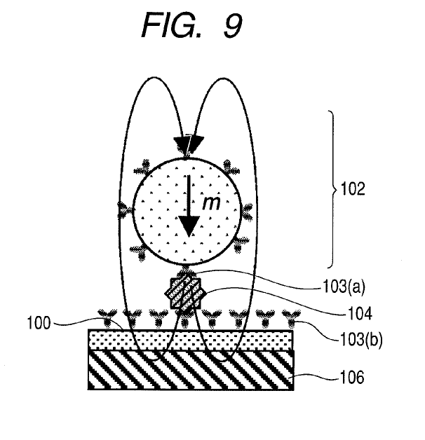

How to fix a target substance onto the TMR sensor will be described with reference to FIG. 9. The following constitution is prepared. That is, the target substance (antigen) 104 is sandwiched between the primary antibody 103(b), which is prepared on the surface 100 of the magnetic sensor, and a secondary antibody 103(a), which is prepared on the surface of Composite particle B102. Since the target substance (antigen) 104 and Composite particle B102 (Example 19) are fixed on the TMR sensor surface 100 in this manner, target substance 104 can be detected by detecting Composite particle B102.

By use of the TMR sensor and fixation a target substance, a prostate specific antigen (PSA) known as a marker of prostate cancer can be detected as a trial according to the following protocol. Note that, the primary antibody 103(b) recognizing PSA is prepared on the TMR sensor surface 100.

(1) a phosphate buffered physiological saline solution (test solution) containing an antigen, PSA, (test sample) is supplied into a channel prepared so as to be in contact with the TMR sensor surface 100 and incubated for 5 minutes.

(2) A phosphate buffered physiological saline solution is supplied through the channel to remove unreacted PSA.

(3) A phosphate buffered physiological saline solution containing Composite particle B whose surface is modified with an anti-PSA antibody (secondary antibody) is supplied into the channel and incubated for 5 minutes.

(4) unreacted labeled antibody is washed away with a phosphate buffered physiological saline solution.

According to the above protocol, Composite particle B102 is fixed onto the TMR sensor surface 100 via the anti-PSA antibody (secondary antibody) 103(a) and the antigen 104 and the primary antibody 103(b). In other words, when antigen 104 is not present in a test sample, Composite particle B102 is not fixed onto the TMR sensor surface 100. Therefore, the presence/absence of the antigen can be detected by detecting the presence/absence of Composite particle B102.

iii) Measurement Procedure

In the same detection manner as in Example 19, the presence/absence or concentration of composite particles fixed on the TMR sensor surface 100 is detected. Based on the detection, the presence/absence and concentration of the target substance, antigen 104, can be detected.

Note that, in this example, Section ii) above, the case where a single TMR sensor and a single channel are formed is described. However, if a plurality of detection units and one or more channels are prepared such that different antigen-antibody reactions occur in individual detection units, a plurality of antigens can be detected at a time.

### INDUSTRIAL APPLICABILITY

The composite particles of the present invention are small in particle diameter, excellent in mono-dispersibility, high in magnetic-substance content, large in saturation magnetization per particle and excellent in dispersion stability and has a non-specific adsorption suppressibility. Therefore, the composite particles can be used as composite particles that can be applied to a wide variety of industrial fields including medical materials, particularly, magnetic particles suitable for a magnetic biosensor of magnetically detecting the presence/absence or concentration of a target substance in a test solution.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-154646, filed Jun. 12, 2008, which is hereby incorporated by reference in its entirety.

1. A method for producing composite particles comprising:
(1) mixing a first liquid and particles to prepare a mixture solution;
(2) mixing the mixture solution and a second liquid to prepare an emulsion containing a dispersoid formed of the first liquid and the particles;
(3) mixing a polymer compound with the emulsion; and
(4) fractionating the emulsion to extract the first liquid from the dispersoid to produce the composite particles each containing the particles and the polymer compound, wherein the dispersoid has a single-peak particle size distribution and a dispersity index (Dhw/Dhn) calculated from a number-average hydrodynamic particle size (Dhn) and a weight-average hydrodynamic particle size (Dhw) is 1.5 or less.

2. The method for producing composite particles according to claim 1, wherein the emulsion is a mini-emulsion.

3. The method for producing composite particles according to claim 1, wherein the first liquid is an organic solvent insoluble in the second liquid or a monomer.

4. The method for producing composite particles according to claim 1, wherein the polymer compound varies between an insoluble state and a soluble state depending upon the pH of the second liquid.

5. The method for producing composite particles according to claim 1, further comprising changing a pH of the emulsion from a pH at which the polymer compound is soluble in the second liquid to a pH at which the polymer compound is insoluble.

6. The method for producing composite particles according to claim 1, wherein the polymer compound is an amphoteric polymer compound having a hydrophobic site and a hydrophilic site.

7. The method for producing composite particles according to claim 1, wherein the particles are particles containing a magnetic substance and a weight-average dry particle size (Dw) of the magnetic substance is 20 nm or less.

8. Composite particles each having a structure in which substantially spherical multinuclear particles formed of a plurality of magnetic substances are surrounded by a film-state polymer compound, wherein a polydispersity index (Dw/Dn) calculated from a number-average dry particle size (Dn) and a weight-average dry particle size (Dw) of the composite particles is 1.2 or less; the weight-average dry particle size (Dw) of the composite particles falls within a range of 50 nm to 300 nm; a content of the magnetic substances in the composite particles is not less than 50 wt % to not more than 90 wt %; and a weight-average dry particle size (Dw) of the substantially spherical multinuclear particles formed of the magnetic substances is 20 nm or less.

9. The composite particles according to claim 8, wherein the composite particles at least partly have a hollow structure.

10. A dispersion solution prepared by dispersing composite particles in water or an aqueous solution, wherein the dispersion solution has a single-peak particle size distribution and a dispersity index (Dhw/Dhn) calculated from a number-average hydrodynamic particle size (Dhn) and a weight-average hydrodynamic particle size (Dhw) is 1.2 or less; the composite particles each have a structure in which substantially spherical multinuclear particles formed of a plurality of magnetic substances are surrounded by a film-state polymer compound; a polydispersity index (Dw/Dn) calculated from a number-average dry particle size (Dn) and a weight-average dry particle size (Dw) of the composite particles is 1.2 or less; the weight-average dry particle size (Dw) of the composite particles falls within a range of 50 nm to 300 nm; a content of the magnetic substances in the composite particles is not less than 50 wt % to not more than 90 wt %; and a weight-average dry particle size (Dw) of the substantially spherical multinuclear particles formed of the magnetic substances is 20 nm or less.

11. A magnetic biosensing apparatus having the composite particles A according to claim 8 and a magnetic sensor.

12. A magnetic biosensing method comprising binding a target substance trapping substance to a surface of the composite particles A according to claim 8 to obtain composite particles B capable of trapping the target substance; bringing the composite particles B capable of trapping the target substance into contact with a sample to trap the target substance in the sample; and
detecting the composite particles B trapping the target substance by the magnetic sensor to determine the presence/absence or concentration of the target substance in the sample.

13. The magnetic biosensing method according to claim 12, comprising fixing the composite particles B trapping the target substance to the surface of the magnetic sensor and applying a static magnetic field to the composite particles B fixed on the surface of the magnetic sensor.
